(19) United States
(12) Patent Application Publication
(10) Pub. No.: US 2025/0288415 A1
(43) Pub. Date: Sep. 18, 2025

(54) PROSTHETIC HEART VALVES WITH COMMISSURE COUPLING ELEMENTS

(71) Applicant: EDWARDS LIFESCIENCES CORPORATION, Irvine, CA (US)

(72) Inventors: Michael Bukin, Pardes Hanna (IL); Nikolai Gurovich, Hadera (IL); Elena Sherman, Pardes Hana (IL)

(73) Assignee: EDWARDS LIFESCIENCES CORPORATION, Irvine, CA (US)

(21) Appl. No.: 19/221,749

(22) Filed: May 29, 2025

Related U.S. Application Data
(63) Continuation of application No. PCT/US2023/082634, filed on Dec. 6, 2023.
(60) Provisional application No. 63/432,029, filed on Dec. 12, 2022.

(51) Int. Cl. A61F 2/24

(52) U.S. Cl. CPC A61F 2/243; A61F 2/2418; A61F 2220/0025; A61F 2230/0006; A61F 2230/0026

(57) ABSTRACT

A prosthetic valve includes an annular frame and a valvular structure mounted within the annular frame. The valvular structure comprises a plurality of leaflets and a plurality of offset elements. Each leaflet includes a leaflet free edge that is offset in an axial direction from the commissures by upper material portions that are unattached to the frame. Commissures are formed by primary tabs on the side edges of adjacent leaflets attached to secondary tabs on opposing ends of the offset elements. A bridge portion of the offset elements extends over an end portion of the leaflet free edges of the adjacent leaflets and is moveable between a neutral position and a radially outward shifted position. An articulation axis between the adjacent leaflets is offset greater distance from the frame when the bridge portion is in the neutral position relative to when the bridge portion is in the radially outward shifted position.

### CROSS-REFERENCE TO RELATED APPLICATIONS

This application is a continuation of PCT Application No. PCT/US2023/082634, filed Dec. 6, 2023, which claims the benefit of U.S. Provisional Patent Application No. 63/432,029, filed Dec. 12, 2022, which are each incorporated by reference herein in their entireties.

### FIELD

The present disclosure relates to valvular structures and frames for prosthetic valves and to delivery apparatus and methods for implanting prosthetic valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans.

Percutaneous and minimally invasive surgical approaches are used in various procedures to deliver prosthetic medical devices to locations inside the body that are not readily accessible by surgery or where access without surgery is desirable. In one specific example, a prosthetic valve can be mounted in a crimped state on the distal end of a delivery device and advanced through the patient's vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery device so that the prosthetic valve can self-expand to its functional size.

The prosthetic valve can include a plurality of leaflets that cycle between closed and open states during the diastolic and systolic phases of the heart. The leaflets should desirably minimize pressure gradients across the valve when open during the systolic phase and properly coapt with each other during diastole.

### SUMMARY

Described herein are prosthetic heart valves, delivery apparatus, and methods for implanting prosthetic heart valves. The disclosed prosthetic heart valves, delivery apparatus, and methods can, for example, provide a valvular structure that can be adjusted to alter an offset distance of leaflet articulation axes relative to the frame in order to enable use of the prosthetic heart valve across a wider range of valve diameters. As such, the devices and methods disclosed herein can, among other things, overcome one or more of the deficiencies of typical prosthetic heart valves and their delivery apparatus and methods of use.

In some examples, a prosthetic valve comprises: a radially compressible and expandable annular frame; and a valvular structure disposed inside of the frame.

In some examples, the valvular structure can comprise a plurality of leaflets and a plurality of offset elements arranged to form a plurality of commissures, each leaflet including a coaptation edge.

In some examples, each commissure can include one of the offset elements wrapped over a portion of the coaptation edges of a pair of adjacent leaflets such that an articulation axis of the adjacent leaflets is offset radially inwardly from an inner surface of the frame.

In some examples, the offset elements can be moveable relative to the annular frame from a neutral position to a radially outward shifted position.

In some examples, the prosthetic valve can be radially expandable to a first expanded diameter and a second expanded diameter greater than the first expanded diameter.

In some examples, when the prosthetic valve is expanded to the first expanded diameter, the offset elements can be in the neutral position and, when the prosthetic valve is expanded to the second expanded diameter, the offset elements can be in the outward shifted position.

In some examples, the articulation axis of each commissure can be closer to the inner surface of the frame when the offset elements are in the outward shifted position.

In some examples, the offset elements can include a moveable bridge portion and opposing end portions, wherein the opposing end portions are attached to the adjacent leaflets.

In some examples, each of the commissures can further include a first primary commissure tab of a first leaflet in the pair of adjacent leaflets, and a second primary commissure tab of a second leaflet in the pair of adjacent leaflets.

In some examples, the first and second primary commissure tabs can extend through a commissure window of the frame from an interior to an exterior of the frame.

In some examples, each offset element can include a first secondary commissure tab extending from one end portion of the offset element and a second secondary commissure tab extending from an opposing end portion of the offset element.

In some examples, the first secondary commissure tab can extend over an interior surface of a first strut of the commissure window, and the second secondary commissure tab can extend over an interior surface of a second strut of the commissure window.

In some examples, the first primary commissure tab can extend over an exterior surface of a first strut of the commissure window, and the second primary commissure tab can extend over an exterior surface of a second strut of the commissure window.

In some examples, the first primary commissure tab can be sutured to the first secondary commissure tab, and the second primary commissure tab can be sutured to the second secondary commissure tab.

In some examples, the bridge portion can have a trapezoidal shape.

In some examples, the bridge portion can have an interior edge, an exterior edge parallel to the interior edge, a first inclined edge, and a second inclined edge, wherein the interior edge is shorter than the exterior edge.

In some examples, the first and second secondary tabs can extend outwardly relative to the exterior edge of the bridge portion.

In some examples, the prosthetic valve can have a minimum operational diameter in a range of 20 mm to 40 mm.

In some example, the prosthetic valve have an effective operating range from the minimum operational diameter up to an additional 4 mm.

In some examples, when the offset elements are in the neutral position, the prosthetic valve can have a smaller effective outflow area relative to when the offset elements are in the radially outward shifted position.

In some examples, when the offset elements are in the radially outward shifted position, the prosthetic valve can have a larger effective outflow area relative to when the offset elements are in the neutral position.

In some examples, a prosthetic valve can include one or more of the features in Examples 1-66 or 73-88 below.

In some examples, a method of implanting a prosthetic valve can include: inserting a distal end of a delivery apparatus into a vasculature of a patient, the distal end of the delivery apparatus having the prosthetic valve coupled thereto in a radially compressed state, the prosthetic valve comprising an annular frame and a valvular structure, the valvular structure comprising a plurality of leaflets and offset elements arranged to form a plurality of commissures, wherein the commissures form respective articulation axes for the leaflets that are offset from the annular frame by the offset elements, wherein at each commissure, one of the offset elements extends over free edges of a pair of adjacent leaflets; advancing the prosthetic valve to a selected implantation site; and radially expanding the prosthetic valve from the radially compressed state to a radially expanded state.

In some examples, the offset elements can be in a neutral position after the prosthetic valve is radially expanded.

In some examples, the method can include further radially expanding the prosthetic valve to a further radially expanded state, which causes the offset elements to move from the neutral position to a radially outward shifted position.

In some examples, in the radially outward shifted position of the offset elements, the articulation axes are closer to the frame.

In some examples, in the radially outward shifted position of the offset elements, the effective outflow area of the prosthetic valve is increased relative to when the offset elements are in the neutral position.

In some examples, the above method(s) can be performed on a living animal or on a simulation, such as on a cadaver, cadaver heart, anthropomorphic ghost, simulator (for example, with body parts, heart, tissue, etc. being simulated).

In some examples, a method can include one or more of the features in Examples 67-72 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

The following is a description of the figures in the accompanying drawings.

FIG. 1A is a perspective view of one example of a prosthetic valve including a frame and a plurality of leaflets attached to the frame.

FIG. 1B is a perspective view of the prosthetic valve of FIG. 1A with an outer skirt disposed around the frame.

FIG. 2A is a perspective view of a frame for the prosthetic valve of FIG. 1A. FIG. 2B is a front portion of the frame shown in FIG. 2A.

FIG. 3 is a side elevation view of a delivery apparatus for a prosthetic device, such as a prosthetic valve, according to one example.

FIG. 4 is a perspective view of a portion of an actuator of the prosthetic device of FIGS. 1A-2B and an actuator assembly of a delivery apparatus, according to one example.

FIG. 5 is a perspective view of the actuator and actuator assembly of FIG. 4 with the actuator assembly physically coupled to the actuator.

FIG. 6A is a perspective view of a partially assembled valvular structure including leaflets, according to one example.

FIG. 6B is a perspective view of the valvular structure of FIG. 6A including offset elements attached to the leaflets, according to one example.

FIG. 7A is a flattened view of one the offset elements of the exemplary valvular structure of FIGS. 6A and 6B.

FIG. 7B a flattened view of one of the leaflets of the exemplary valvular structure of FIGS. 6A and 6B.

FIG. 7C is a flattened view of the leaflet of FIG. 7.B and the two offset elements of FIG. 7A where the offset elements are folded at each corner of the leaflet such that the tabs of the offset elements align with the tabs of the leaflet.

FIG. 8A is a perspective view of two adjacent leaflet elements and one offset element folded over the corners of the adjacent leaflets where the offset element is in a neutral position, according to one example.

FIG. 8B is a perspective view of the exemplary offset element and adjacent leaflets of FIG. 8A, showing an offset distance when the offset element is in the neutral position and a distance the offset element can be shifted when moved to a radially outward shifted position.

FIG. 8C is a flattened view a leaflet and an offset element shown in FIGS. 8A and 8B, showing deformation of the leaflet and the offset element during diastole.

FIG. 9 is a schematic illustration a commissure of a valvular structure formed by insertion of tabs of adjacent leaflets and tabs of an offset element through a commissure window of a frame of a prosthetic valve, according to one example.

FIG. 10A is an interior perspective view of a commissure of a prosthetic valve including an offset element, according to one example.

FIG. 10B is an exterior side view of the commissure of the prosthetic valve of FIG. 10A.

FIG. 11A is a top view of the commissure of the prosthetic valve of FIG. 10A during systole, where the offset element is in a neutral state or position.

FIG. 11B is a top view of the commissure of the prosthetic valve of FIG. 10A during diastole, where the offset element is shifted radially outward relative to the neutral state or position.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

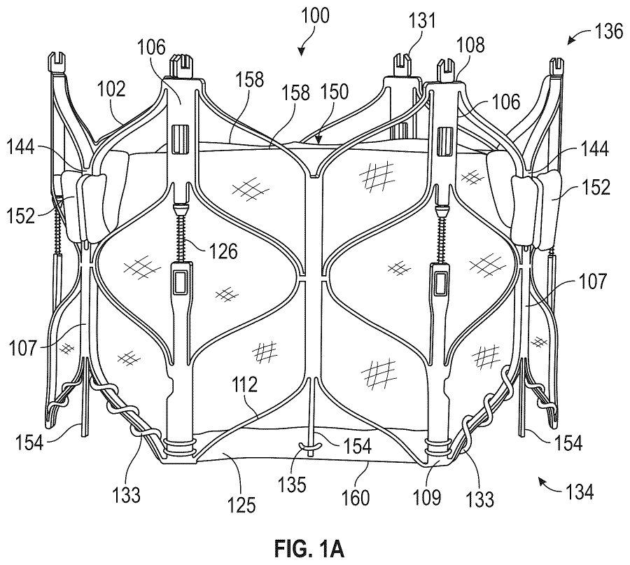

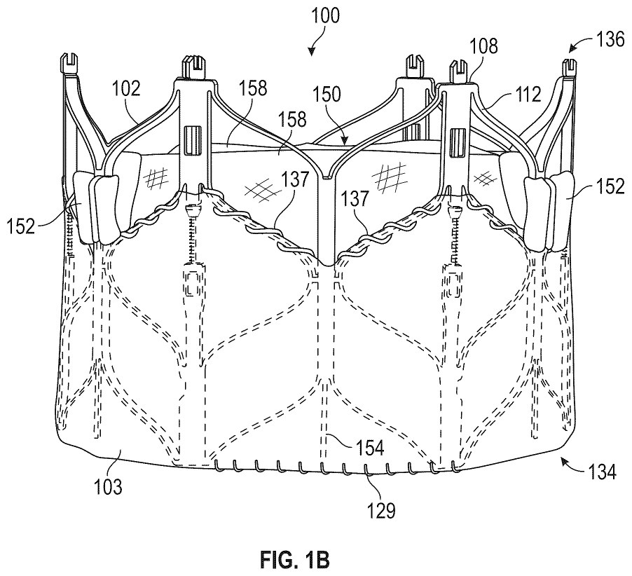

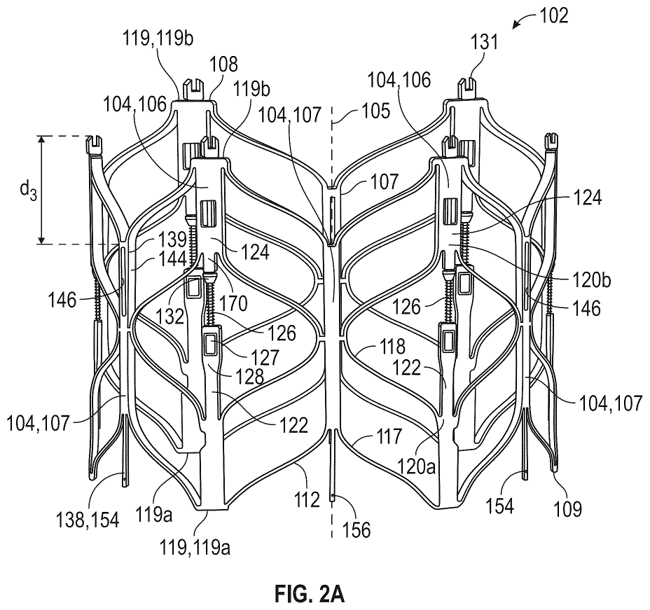

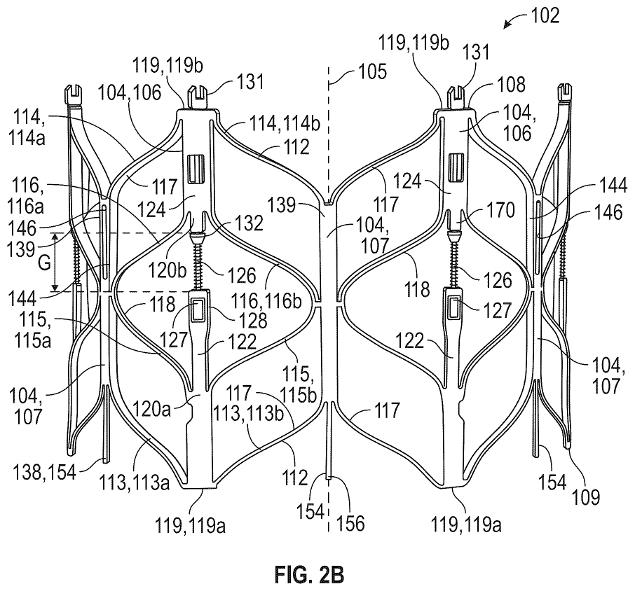

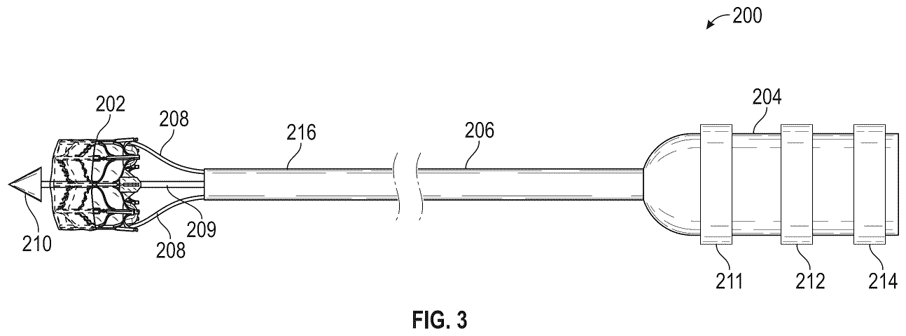

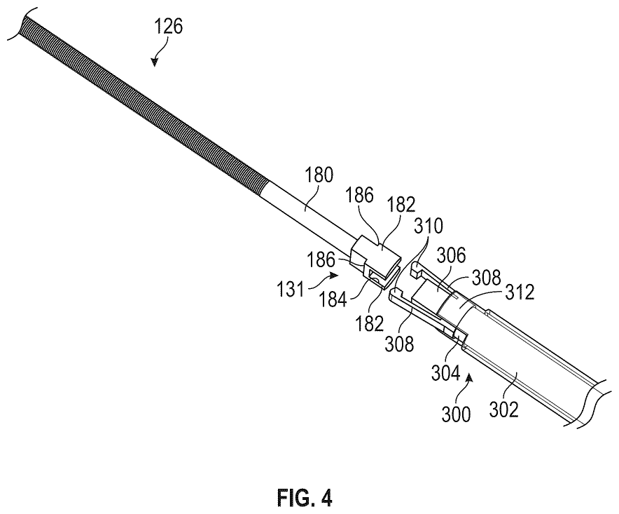

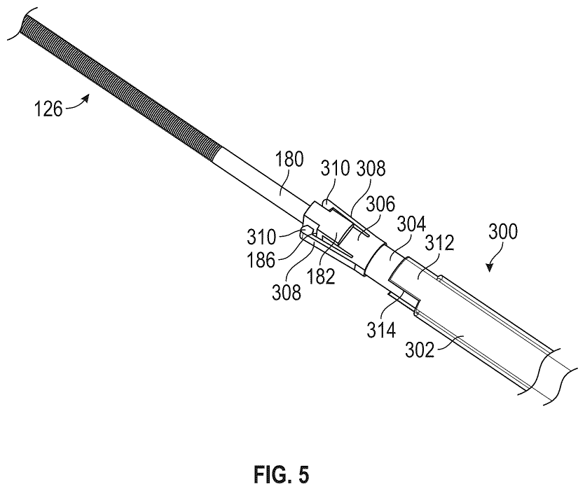

FIGS. 1A-2B illustrate an exemplary prosthetic device (for example, prosthetic heart valve) that can be advanced through a patient's vasculature, such as to a native heart valve, by a delivery apparatus, such as the exemplary delivery apparatus shown in FIG. 3. The frame of the prosthetic heart valve can include one or more mechanical expansion and locking mechanisms that can be integrated into the frame-specifically, into axially extending posts of the frame. The mechanical expansion and/or locking mechanisms, such as the exemplary mechanisms shown in FIGS. 4 and 5, can be removably coupled to, and/or actuated by, the delivery apparatus to radially expand the prosthetic heart valve and lock the prosthetic heart valve in one or more radially expanded states.

As noted above, a prosthetic valve can include a plurality of leaflets that cycle between closed and open states during the diastolic and systolic phases of the heart. The leaflets should desirably minimize pressure gradients across the valve when open during the systolic phase and properly coapt with each other during diastole. However, achieving this operation principle with a single leaflet design over a wide range of valve diameters (for example, 20 mm to 30 mm) is challenging.

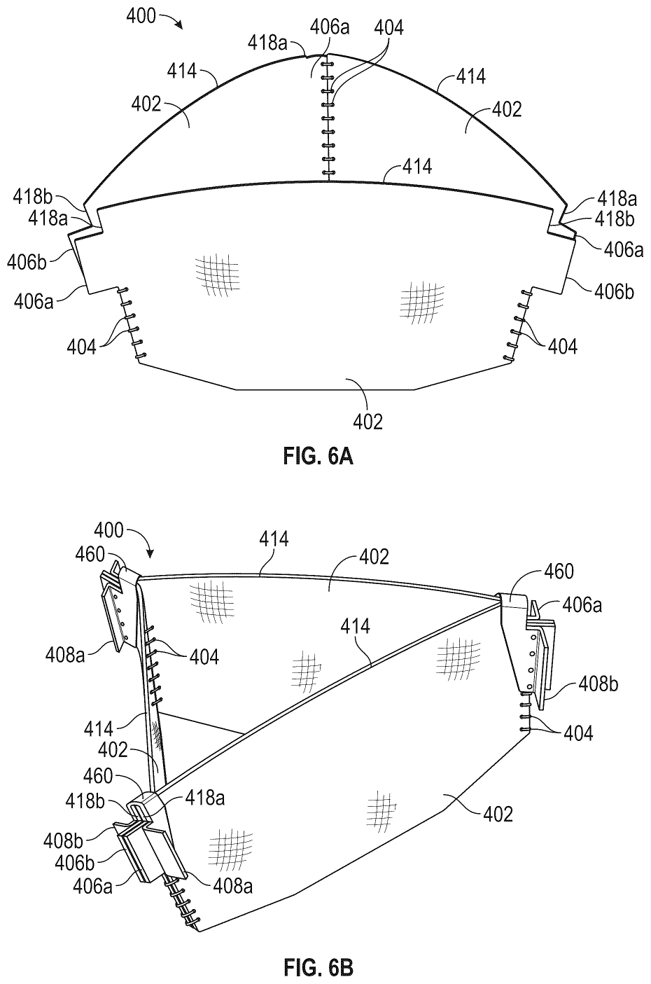

FIGS. 6A-11B illustrate an exemplary valvular structure for a prosthetic device, such as the prosthetic heart valve shown in FIGS. 1A-2B or other prosthetic heart valves (for example, self-expandable prosthetic heart valves). In some examples, the valvular structure can include a plurality of leaflets (for example, three leaflets) that are attached the to the frame at or proximate to the inflow end of the frame, and include free edges at or proximate to the outflow end of the frame. The leaflet free edges can be configured to alternately coapt and separate in order to close and open the prosthetic valve. For example, during ventricular systole, the free edges of the valvular structure can separate to open the valve and enable blood to flow therethrough. As pressure in the ventricle decreases, the leaflet free edges can coapt to close the prosthetic valve (during ventricular diastole).

In some examples, the leaflets can each be attached to adjacent leaflets at their side edges. Further, the outflow end corners of the side edges of the adjacent leaflets can be attached to the frame to form commissures. During opening and closing of the prosthetic valve, articulation or pivot of the adjacent (paired) leaflets can occur around an articulation axis at the commissures. Over a period of implantation of the prosthetic valve, repeated articulation of the leaflets can cause wear or damage to the leaflets, particularly if the leaflets contact or rub against the frame during opening and closing of the leaflet free edges. Accordingly, it can be desirable to offset the articulation axes of the leaflets from the frame to limit, prevent, or minimize contact between the leaflet free edges and the frame during operation of the prosthetic valve. For example, the articulation axes can be offset radially inwardly relative to the frame by a specified offset distance.

A desired offset distance, however, may differ depending on a degree of radial expansion of the prosthetic valve, where the degree of radial expansion is dependent on, for example, anatomy of a patient, a disease condition of the native valve, a location of implantation of the prosthetic valve, and/or other factors. If the offset distance is too small, the leaflets can undergo wear or damage by repeated contact with the frame. If the offset distance is too large, the effective outflow area (EOA) of the leaflets in an open configuration can be reduced and undesirably increase pressure gradients across the prosthetic valve. Thus, a desired offset distance may be a distance where the articulation axes of the leaflets are positioned to prevent or reduce contact between the leaflets and the frame during opening and closing of the prosthetic valve, but also enable the leaflets to open to have an EOA that maintains appropriate pressure gradients for effective heart function when the prosthetic valve is implanted within the heart.

Accordingly, the exemplary valvular structures disclosed herein can include moveable, adjustable, slidable, and/or deformable offset elements disposed at the commissures formed by the adjacent leaflets. In some examples, the offset elements can be folded over or extended over the corners of the free edges of adjacent leaflets and can be attached to the adjacent leaflets at overlapping or paired tab portions. A central bridge portion of each of the offset elements can offset the articulation axes of the respective commissure a distance that corresponds to a width of the bridge portion when the offset element is in a neutral state. The bridge portion can be selectively shifted radially outward to decrease the offset distance.

For example, when a prosthetic valve including a valvular structure having offset elements is radially expanded to a lesser degree (for example, where the prosthetic valve is radially expanded to a relatively smaller diameter), the offset elements can remain in a neutral state or position, where the offset distance corresponds to the width of the bridge portion. Alternatively, in another example, when a prosthetic valve including a valvular structure having offset elements is radially expanded to a greater degree (for example, where the prosthetic valve is expanded to a relatively larger diameter), the offset elements can be moved or deformed into a radially outward shifted state or position, where the offset distance is reduced relative to the neutral state or position. Specifically, the offset distance can be equal to the width of the bridge portion less the distance that the offset clement is radially shifted outward.

Thus, the exemplary valvular structures disclosed herein can, when the prosthetic valve is radially expanded to a lesser diameter and the offset elements are maintained in the neutral position or state, prevent or reduce wear or damage to the leaflets by sufficiently offsetting of the leaflet free edges from the frame of the prosthetic valve. Further, the exemplary valvular structures disclosed herein can, when the prosthetic valve is radially expanded to a greater diameter and the offset elements are moved or deformed to the radially outward shifted state or position, enable the leaflets to have an increased EOA for the valve and reduce pressure gradients across the valve while still preventing or reducing wear or damage to the leaflets by sufficiently offsetting of the leaflet free edges from the frame.

### Examples of the Disclosed Technology

FIGS. 1A-2B show an exemplary prosthetic valve 100, according to one example. In some examples, the prosthetic valves disclosed herein are adapted to be implanted in the native aortic annulus, although in some examples they can additionally or alternatively be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

FIGS. 1A-2B illustrate an example prosthetic valve 100 (which also may be referred to herein as “prosthetic heart valve 100”) having a frame 102. FIGS. 2A-2B show the frame 102 by itself, while FIGS. 1A-1B show the frame 102 with a valvular structure 150 (which can comprise leaflets 158, as described further below) mounted within and to the annular frame 102. FIG. 1B additionally shows an optional skirt assembly comprising an outer skirt 103. While only one side of the frame 102 is depicted in FIG. 2B, it should be appreciated that the frame 102 forms an annular structure having an opposite side that is substantially identical to the portion shown in FIG. 1B, as shown in FIGS. 1A-2A.

As shown in FIGS. 1A and 1B, the valvular structure 150 is coupled to and supported inside the frame 102. The valvular structure 150 is configured to regulate the flow of blood through the prosthetic valve 100, from an inflow end portion 134 to an outflow end portion 136. The valvular structure 150 can include, for example, a leaflet assembly comprising one or more leaflets 158 made of flexible material. The leaflets 158 can be made from in whole or part, biological material, bio-compatible synthetic materials, or other such materials. Suitable biological material can include, for example, bovine pericardium (or pericardium from other sources). The leaflets 158 can be secured to one another at their adjacent sides to form commissures 152, each of which can be secured to a respective commissure support structure 144 (also referred to herein as “commissure supports”) and/or to other portions of the frame 102, as described in greater detail below.

In the example depicted in FIGS. 1A and 1B, the valvular structure 150 includes three leaflets 158, which can be arranged to collapse in a tricuspid arrangement. Each leaflet 158 can have an inflow edge portion 160 (which can also be referred to as a cusp edge portion) (FIG. 1A). The inflow edge portions 160 of the leaflets 158 can define an undulating, curved scallop edge that generally follows or tracks portions of struts 112 of frame 102 in a circumferential direction when the frame 102 is in the radially expanded configuration. The inflow edge portions 160 of the leaflets 158 can be referred to as a “scallop line.”

The prosthetic valve 100 may include one or more skirts mounted around the frame 102. For example, as shown in FIG. 1B, the prosthetic valve 100 may include an outer skirt 103 mounted around an outer surface of the frame 102. The outer skirt 103 can function as a sealing member for the prosthetic valve 100 by sealing against the tissue of the native valve annulus and helping to reduce paravalvular leakage past the prosthetic valve 100. In some cases, an inner skirt (not shown) may be mounted around an inner surface of the frame 102. The inner skirt can function as a sealing member to prevent or decrease perivalvular leakage, to anchor the leaflets 158 to the frame 102, and/or to protect the leaflets 158 against damage caused by contact with the frame 102 during crimping and during working cycles of the prosthetic valve 100. In some examples, the inflow edge portions 160 of the leaflets 158 can be sutured to the inner skirt generally along the scallop line. The inner skirt can in turn be sutured to adjacent struts 112 of the frame 102. In some examples, as shown in FIG. 1A, the leaflets 158 can be sutured directly to the frame 102 or to a reinforcing member 125 (also referred to as a reinforcing skirt or connecting skirt) in the form of a strip of material (for example, a fabric strip) which is then sutured to the frame 102, along the scallop line via stitches (for example, whip stitches) 133.

The inner and outer skirts and the connecting skirt 125 can be formed from any of various suitable biocompatible materials, including any of various synthetic materials, including fabrics (for example, polyethylene terephthalate fabric) or natural tissue (for example, pericardial tissue). Further details regarding the use of skirts or sealing members in prosthetic valve can be found, for example, in U.S. Patent Publication No. 2020/0352711, which is incorporated herein by reference.

Further details regarding the assembly of the leaflet assembly and the assembly of the leaflets and the skirts to the frame can be found, for example, in U.S. Provisional Application Nos. 63/209,904, filed Jun. 11, 2021, and 63/224,534, filed Jul. 22, 2021, which are incorporated herein by reference. Further details of the construction and function of the frame 102 can be found in International Patent Application No. PCT/US2021/052745, filed Sep. 30, 2021, which is incorporated herein by reference.

The frame 102, which is shown alone and in greater detail in FIGS. 2A and 2B, comprises and inflow end 109, an outflow end 108, and a plurality of axially extending posts 104. The axial direction of the frame 102 is indicated by a longitudinal axis 105, which extends from the inflow end 109 to the outflow end 108 (FIGS. 2A and 2B). Some of the posts 104 can be arranged in pairs of axially aligned first and second struts or posts 122, 124. An actuator 126 (such as the illustrated threaded rod or bolt) can extend through one or more pairs of posts 122, 124 to form an integral expansion and locking mechanism or actuator mechanism 106 configured to radially expand and compress the frame 102, as further described below. One or more of posts 104 can be configured as support posts 107.

The actuator mechanisms 106 (which can be used to radially expand and/or radially compress the prosthetic valve 100) can be integrated into the frame 102 of the prosthetic valve 100, thereby reducing the crimp profile and/or bulk of the prosthetic valve 100. Integrating the actuator mechanisms 106 (which can also be referred to herein as “expansion and locking mechanisms”) into the frame 102 can also simplify the design of the prosthetic valve 100, making the prosthetic valve 100 less costly and/or easier to manufacture. In the illustrated example, an actuator 126 extends through each pair of axially aligned posts 122, 124. In some examples, one or more of the pairs of posts 122, 124 can be without a corresponding actuator.

The posts 104 can be coupled together by a plurality of circumferentially extending link members or struts 112. Each strut 112 extends circumferentially between adjacent posts 104 to connect all of the axially extending posts 104. As one example, the prosthetic valve 100 can include equal numbers of support posts 107 and pairs of actuator posts 122, 124 and the pairs of posts 122, 124 and the support posts 107 can be arranged in an alternating order such that each strut 112 is positioned between one of the pairs of posts 122, 124 and one of the support posts 107 (i.e., each strut 112 can be coupled on one end to one of the posts 122, 124 and can be coupled on the other end to one of the support posts 107). However, the prosthetic valve 100 can include different numbers of support posts 107 and pairs of posts 122, 124 and/or the pairs of posts 122, 124 and the support posts 107 can be arranged in a non-alternating order, in some examples.

As illustrated in FIG. 2B, the struts 112 can include a first row of struts 113 at or near the inflow end 109 of the prosthetic valve 100, a second row of struts 114 at or near the outflow end 108 of the prosthetic valve 100, and third and fourth rows of struts 115, 116, respectively, positioned axially between the first and second rows of struts 113, 114. The struts 112 can form and/or define a plurality of cells (i.e., openings) in the frame 102. For example, the struts 113, 114, 115, and 116 can at least partially form and/or define a plurality of first cells 117 and a plurality of second cells 118 that extend circumferentially around the frame 102. Each first cell 117 can be formed by two struts 113a, 113b of the first row of struts 113, two struts 114a, 114b of the second row of struts 114, and two of the support posts 107. Each second cell 118 can be formed by two struts 115a, 115b of the third row of struts 115 and two struts 116a, 116b of the fourth row of struts 116. As illustrated in FIGS. 2A and 2B, each second cell 118 can be disposed within one of the first cells 117 (i.e., the struts 115a-116b forming the second cells 118 are disposed between the struts forming the first cells 117 (i.e., the struts 113a, 113b and the struts 114a, 114b), closer to an axial midline of the frame 102 than the struts 113a-114b).

As illustrated in FIGS. 2A and 2B, the struts 112 of frame 102 can comprise a curved shape. Each first cell 117 can have an axially-extending hexagonal shape including first and second apices 119 (for example, an inflow apex 119a and an outflow apex 119b). In examples where the delivery apparatus is releasably connected to the outflow apices 119b (as described below), each inflow apex 119a can be referred to as a “distal apex” and each outflow apex 119b can be referred to as a “proximal apex”. Each second cell 118 can have a diamond shape including first and second apices 120 (for example, distal apex 120a and proximal apex 120b). In some examples, the frame 102 comprises six first cells 117 extending circumferentially in a row, six second cells 118 extending circumferentially in a row within the six first cells 117, and twelve posts 104. However, in some examples, the frame 102 can comprise a greater or fewer number of first cells 117 and a correspondingly greater or fewer number of second cells 118 and posts 104.

As noted above, some of the posts 104 can be arranged in pairs of first and second posts 122, 124. The posts 122, 124 are aligned with each other along the length of the frame 102 and are axially separated from one another by a gap G (FIG. 2B) (those with actuators 126 can be referred to as actuator posts or actuator struts). Each first post 122 (i.e., the lower post shown in FIGS. 2A and 2B) can extend axially from the inflow end 109 of the prosthetic valve 100 toward the second post 124, and the second post 124 (i.e., the upper post shown in FIGS. 2A and 2B) can extend axially from the outflow end 108 of the prosthetic valve 100 toward the first post 122. For example, each first post 122 can be connected to and extend from an inflow apex 119a and each second post 124 can be connected to and extend from an outflow apex 119b. Each first post 122 and the second post 124 can include an inner bore configured to receive a portion of an actuator member, such as in the form of a substantially straight threaded rod 126 (or bolt) as shown in the illustrated example. The threaded rod 126 also may be referred to herein as actuator 126, actuator member 126, and/or screw actuator 126. In examples where the delivery apparatus can be releasably connected to the outflow end 108 of the frame 102, the first posts 122 can be referred to as distal posts or distal axial struts and the second posts 124 can be referred to as proximal posts or proximal axial struts.

Each threaded rod 126 extends axially through a corresponding first post 122 and second post 124. Each threaded rod 126 also extends through a bore of a nut 127 captured within a slot or window formed in an end portion 128 of the first post 122. The threaded rod 126 has external threads that engage internal threads of the bore of the nut 127. The inner bore of the second post 124 (through which the threaded rod 126 extends) can have a smooth and/or non-threaded inner surface to allow the threaded rod 126 to slide freely within the bore. Rotation of the threaded rod 126 relative to the nut 127 produces radial expansion and compression of the frame 102, as further described below.

In some examples, the threaded rod 126 can extend past the nut 127 toward the inflow end 109 of the frame 102 into the inner bore of the first post 122. The nut 127 can be held in a fixed position relative to the first post 122 such that the nut 127 does not rotate relative to the first post 122. In this way, whenever the threaded rod 126 is rotated (for example, by a physician) the threaded rod 126 can rotate relative to both the nut 127 and the first post 122. The engagement of the external threads of the threaded rod 126 and the internal threads of the nut 127 prevent the rod 126 from moving axially relative to the nut 127 and the first post 122 unless the threaded rod 126 is rotated relative to the nut 127. Thus, the threaded rod 126 can be retained or held by the nut 127 and can only be moved relative to the nut 127 and/or the first post 122 by rotating the threaded rod 126 relative to the nut 127 and/or the first post 122. In some examples, in lieu of using the nut 127, at least a portion of the inner bore of the first post 122 can be threaded. For example, the bore along the end portion 128 of the first post 122 can comprise inner threads that engage the external threaded rod 126 such that rotation of the threaded rod causes the threaded rod 126 to move axially relative to the first post 122.

When a threaded rod 126 extends through and/or is otherwise coupled to a pair of axially aligned posts 122, 124, the pair of axially aligned posts 122, 124 and the threaded rod 126 can serve as one of the expansion and locking mechanisms 106. In some examples, a threaded rod 126 can extend through each pair of axially aligned posts 122, 124 so that all of the posts 122, 124 (with their corresponding rods 126) serve as expansion and locking mechanisms 106. As just one example, the prosthetic valve 100 can include six pairs of posts 122, 124, and each of the six pairs of posts 122, 124 with their corresponding rods 126 can be configured as one of the expansion and locking mechanisms 106 for a total of six expansion and locking mechanisms 106. In some examples, not all pairs of posts 122, 124 need be expansion and locking mechanisms (i.e., actuators). If a pair of posts 122, 124 is not used as an expansion and locking mechanism, a threaded rod 126 need not extend through the posts 122, 124 of that pair.

The threaded rod 126 can be rotated relative to the nut 127, the first post 122, and the second post 124 to axially foreshorten and/or axially elongate the frame 102, thereby radially expanding and/or radially compressing, respectively, the frame 102 (and therefore the prosthetic valve 100). For example, when the threaded rod 126 is rotated relative to the nut 127, the first post 122, and the second post 124, the first and second posts 122, 124 can move axially relative to one another, thereby widening or narrowing the gap G (FIG. 2B) separating the posts 122, 124, and thereby radially compressing or radially expanding the prosthetic valve 100, respectively. Thus, the gap G (FIG. 2B) between the first and second posts 122, 124 narrows as the frame 102 is radially expanded and widens as the frame 102 is radially compressed.

The threaded rod 126 can extend proximally past the proximal end of the second post 124 and can include a head portion 131 at its proximal end that can serve at least two functions. First, the head portion 131 can removably or releasably couple the threaded rod 126 to a respective actuator assembly of a delivery apparatus that can be used to radially expand and/or radially compress the prosthetic valve 100 (for example, the delivery apparatus 200 of FIG. 3, as described below). Second, the head portion 131 can prevent the second post 124 from moving proximally relative to the threaded rod 126 and can apply a distally directed force to the second post 124, such as when radially expanding the prosthetic valve 100. For example, the head portion 131 can have a width greater than a diameter of the inner bore of the second post 124 such that the head portion 131 is prevented from moving into the inner bore of the second post 124. Thus, as the threaded rod 126 is threaded farther into the nut 127, the head portion 131 of the threaded rod 126 draws closer to the nut 127 and the first post 122, thereby drawing the second post 124 towards the first post 122, and thereby axially foreshortening and radially expanding the prosthetic valve 100.

The threaded rod 126 also can include a stopper 132 (for example, in the form of a nut, washer or flange) disposed thereon. The stopper 132 can be disposed on the threaded rod 126 such that it sits within the gap G. Further, the stopper 132 can be integrally formed on or fixedly coupled to the threaded rod 126 such that it does not move relative to the threaded rod 126. Thus, the stopper 132 can remain in a fixed axial position on the threaded rod 126 such that it moves in lockstep with the threaded rod 126.

Rotation of the threaded rod 126 in a first direction (for example, clockwise) can cause corresponding axial movement of the first and second posts 122, 124 toward one another, thereby decreasing the gap G and radially expanding the frame 102, while rotation of the threaded rod 126 in an opposite second direction causes corresponding axial movement of the first and second posts 122, 124 away from one another, thereby increasing the gap G and radially compressing the frame. When the threaded rod 126 is rotated in the first direction, the head portion 131 of the rod 126 bears against an adjacent surface of the frame (for example, an outflow apex 119b), while the nut 127 and the first post 122 travel proximally along the threaded rod 126 toward the second post 124, thereby radially expanding the frame. As the frame 102 moves from a compressed configuration to an expanded configuration, the gap G between the first and second posts 122, 124 can narrow.

When the threaded rod 126 is rotated in the second direction, the threaded rod 126 and the stopper 132 move toward the outflow end 108 of the frame until the stopper 132 abuts the inflow end 170 of the second post 124 (as shown in FIGS. 2A and 2B). Upon further rotation of the rod 126 in the second direction, the stopper 132 can apply a proximally directed force to the second post 124 to radially compress the frame 102. For example, during crimping/radial compression of the prosthetic valve 100, the threaded rod 126 can be rotated in the second direction (for example, counterclockwise) causing the stopper 132 to push against (i.e., provide a proximally directed force to) the inflow end 170 of the second post 124, thereby causing the second post 124 to move away from the first post 122, and thereby axially elongating and radially compressing the prosthetic valve 100.

Thus, each of the second posts 124 can slide axially relative to a corresponding one of the first posts 122 but can be axially retained and/or restrained between the head portion 131 of a threaded rod 126 and a stopper 132. That is, each second post 124 can be restrained at its proximal end by the head portion 131 of the threaded rod 126 and at its distal end by the stopper 132. In this way, the head portion 131 can apply a distally directed force to the second post 124 to radially expand the prosthetic valve 100 while the stopper 132 can apply a proximally directed force to the second post 124 to radially compress the prosthetic valve 100. As explained above, radially expanding the prosthetic valve 100 axially foreshortens the prosthetic valve 100, causing an inflow end portion 134 and outflow end portion 136 of the prosthetic valve 100 (FIGS. 1A and 1B) to move towards one another axially, while radially compressing the prosthetic valve 100 axially elongates the prosthetic valve 100, causing the inflow and outflow end portions 134, 136 to move away from one another axially.

In some examples, the threaded rod 126 can be fixed against axial movement relative to the second post 124 (and the stopper 132 can be omitted) such that rotation of the threaded rod 126 in the first direction produces proximal movement of the nut 127 and radial expansion of the frame 102 and rotation of the threaded rod 126 in the second direction produces distal movement of the nut 127 and radial compression of the frame 102.

As also introduced above, some of the posts 104 can be configured as support posts 107. As shown in FIGS. 2A and 2B, the support posts 107 can extend axially between the inflow and outflow ends 109, 108 of the frame 102 and each can have an inflow end portion 138 and an outflow end portion 139. The outflow end portion 139 of one or more support posts 107 can include a commissure support structure or member 144. The commissure support structure 144 can comprise strut portions defining a commissure opening 146 therein.

The commissure opening 146 (which can also be referred to herein as a “commissure window 146”) can extend radially through a thickness of the support post 107 and can be configured to accept a portion of a valvular structure 150 (for example, a commissure 152) to couple the valvular structure 150 to the frame 102. For example, each commissure 152 can be mounted to a respective commissure support structure 144, such as by inserting a pair of commissure tabs of adjacent leaflets 158 through the commissure opening 146 and suturing the commissure tabs to each other and/or the commissure support structure 144. In some examples, the commissure opening 146 can be fully enclosed by the support post 107 such that a portion of the valvular structure 150 can be slid radially through the commissure opening 146, from an interior to an exterior of the frame 102, during assembly. In the illustrated example, the commissure opening 146 has a substantially rectangular shape that is shaped and sized to receive commissure tabs of two adjacent leaflets therethrough. However, in some examples, the commissure opening can have any of various shapes (for example, square, oval, square-oval, triangular, L-shaped, T-shaped, C-shaped, etc.).

The commissure openings 146 are spaced apart about the circumference of frame 102 (or angularly spaced apart about frame 102). The spacing may or may not be even. In one example, the commissure openings 146 are axially offset from the outflow end 108 of the frame 102 by an offset distance d₃ (indicated in FIG. 2A). As an example, the offset distance d₃ may be in a range from 2 mm to 6 mm. In general, the offset distance d₃ should be selected such that when the leaflets are attached to the frame 102 via the commissure openings 146, the free edge portions (for example, outflow edge portions) of the leaflets 158 will not protrude from or past the outflow end 108 of the frame 102.

The frame 102 can comprise any number of support posts 107, any number of which can be configured as commissure support structures 144. For example, the frame 102 can comprise six support posts 107, three of which are configured as commissure support structures 144. However, in some examples, the frame 102 can comprise more or less than six support posts 107 and/or more or less than three commissure support structures 144.

The inflow end portion 138 of each support post 107 can comprise an extension 154 (show as a cantilevered strut in FIGS. 2A and 2B) that extends toward the inflow end 109 of the frame 102. Each extension 154 can comprise an aperture 156 extending radially through a thickness of the extension 154. In some examples, the extension 154 can extend such that an inflow edge of the extension 154 aligns with or substantially aligns with the inflow end 109 of the frame 102. In use, the extension 154 can prevent or mitigate portions of an outer skirt from extending radially inwardly and thereby prevent or mitigate any obstruction of flow through the frame 102 caused by the outer skirt. The extensions 154 can further serve as supports to which portions of the inner and/or outer skirts and/or the leaflets and/or the connecting skirt 125 can be coupled. For example, sutures used to connect the inner and/or outer skirts and/or the leaflets and/or the connecting skirt 125 can be wrapped around the extensions 154 and/or can extend through apertures 156.

As an example, each extension 154 can have an aperture 156 (FIG. 2A) or other features to receive a suture or other attachment material for connecting an adjacent inflow edge portion 160 of a leaflet 158 (FIG. 1A), the outer skirt 103 (in FIG. 1B), the connecting skirt 125, and/or an inner skirt. In some examples, the inflow edge portion 160 of each leaflet 158 can be connected to a corresponding extension via a suture 135 (FIG. 1A).

In some examples, the outer skirt 103 can be mounted around the outer surface of frame 102 as shown in FIG. 1B and the inflow edge of the outer skirt 103 (lower edge in FIG. 1B) can be attached to the connecting skirt 125 and/or the inflow edge portions 160 of the leaflets 158 that have already been secured to frame 102 as well as to the extensions 154 of the frame by sutures 129. The outflow edge of the outer skirt 103 (the upper edge in FIG. 1B) can be attached to selected struts with stitches 137. In implementations where the prosthetic valve includes an inner skirt, the inflow edge of the inner skirt can be secured to the inflow edge portions 160 before securing the cusp edge portions to the frame so that the inner skirt will be between the leaflets and the inner surface of the frame. After the inner skirt and leaflets are secured in place, then the outer skirt can be mounted around the frame as described above.

The frame 102 can be a unitary and/or fastener-free frame that can be constructed from a single piece of material (for example, Nitinol, stainless steel or a cobalt-chromium alloy), such as in the form of a tube. The plurality of cells can be formed by removing portions (for example, via laser cutting) of the single piece of material. The threaded rods 126 can be separately formed and then be inserted through the bores in the second (proximal) posts 124 and threaded into the threaded nuts 127.

In some examples, the frame 102 can be formed from a plastically-expandable material, such as stainless steel or a cobalt-chromium alloy. When the frame is formed from a plastically-expandable material, the prosthetic valve 100 can be placed in a radially compressed state along the distal end portion of a delivery apparatus for insertion into a patient's body. When at the desired implantation site, the frame 102 (and therefore the prosthetic valve 100) can be radially expanded from the radially compressed state to a radially expanded state via actuation of actuation assemblies of the delivery apparatus (as further described below), which rotate the rods 126 to produce expansion of the frame 102. During delivery to the implantation site, the prosthetic valve 100 can be placed inside of a delivery capsule (sheath) to protect against the prosthetic valve contacting the patient's vasculature, such as when the prosthetic valve is advanced through a femoral artery. The capsule can also retain the prosthetic valve in a compressed state having a slightly smaller diameter and crimp profile than may be otherwise possible without a capsule by preventing any recoil (expansion) of the frame once it is crimped onto the delivery apparatus.

In some examples, the frame 102 can be formed from a self-expandable material (for example, Nitinol). When the frame 102 is formed from a self-expandable material, the prosthetic valve can be radially compressed and placed inside the capsule of the delivery apparatus to maintain the prosthetic valve in the radially compressed state while it is being delivered to the implantation site. When at the desired implantation site, the prosthetic valve is deployed or released from the capsule. In some examples, the frame (and therefore the prosthetic valve) can partially self-expand from the radially compressed state to a partially radially expanded state. The frame 102 (and therefore the prosthetic valve 100) can be further radially expanded from the partially expanded state to a further radially expanded state via actuation of actuation assemblies of the delivery apparatus (as further described below), which rotate the rods 126 to produce expansion of the frame. Thus, in some examples, the frame 102 is an at least partially self-expandable frame.

As introduced above, the threaded rods 126 can removably couple the prosthetic valve 100 to actuator assemblies of a delivery apparatus. Referring to FIG. 3, it illustrates an exemplary delivery apparatus 200 for delivering a prosthetic device or valve 202 (for example, prosthetic valve 100) to a desired implantation location. The prosthetic valve 202 can be releasably coupled to the delivery apparatus 200. It should be understood that the delivery apparatus 200 and other delivery apparatuses disclosed herein can be used to implant prosthetic devices other than prosthetic valves, such as stents or grafts.

The delivery apparatus 200 in the illustrated example generally includes a handle 204, a first elongated shaft 206 (which comprises an outer shaft in the illustrated example) extending distally from the handle 204, at least one actuator assembly 208 extending distally through the first shaft 206, a second elongated shaft 209 (which comprises an inner shaft in the illustrated example) extending through the first shaft 206, and a nosecone 210 coupled to a distal end portion of the second shaft 209. The second shaft 209 and the nosecone 210 can define a guidewire lumen for advancing the delivery apparatus through a patient's vasculature over a guidewire. The at least one actuator assembly 208 can be configured to radially expand and/or radially collapse the prosthetic valve 202 when actuated, such as by one or more knobs 211, 212, 214 included on the handle 204 of the delivery apparatus 200.

Though the illustrated example shows two actuator assemblies 208 for purposes of illustration, it should be understood that one actuator assembly 208 can be provided for each actuator (for example, actuator or threaded rod 126) on the prosthetic valve. For example, three actuator assemblies 208 can be provided for a prosthetic valve having three actuators. In some examples, a greater or fewer number of actuator assemblies can be present.

In some examples, a distal end portion 216 of the shaft 206 can be sized to house the prosthetic valve in its radially compressed, delivery state during delivery of the prosthetic valve through the patient's vasculature. In this manner, the distal end portion 216 functions as a delivery sheath or capsule for the prosthetic valve during delivery,

The actuator assemblies 208 can be releasably coupled to the prosthetic valve 202. For example, in the illustrated example, each actuator assembly 208 can be coupled to a respective actuator (for example, threaded rod 126) of the prosthetic valve 202. Each actuator assembly 208 can comprise a support tube and an actuator member. When actuated, the actuator assembly can transmit pushing and/or pulling forces to portions of the prosthetic valve to radially expand and collapse the prosthetic valve as previously described. The actuator assemblies 208 can be at least partially disposed radially within, and extend axially through, one or more lumens of the first shaft 206. For example, the actuator assemblies 208 can extend through a central lumen of the shaft 206 or through separate respective lumens formed in the shaft 206.

The handle 204 of the delivery apparatus 200 can include one or more control mechanisms (for example, knobs or other actuating mechanisms) for controlling different components of the delivery apparatus 200 in order to expand and/or deploy the prosthetic valve 202. For example, in the illustrated example, the handle 204 comprises first, second, and third knobs 211, 212, and 214, respectively.

The first knob 211 can be a rotatable knob configured to produce axial movement of the first shaft 206 relative to the prosthetic valve 202 in the distal and/or proximal directions in order to deploy the prosthetic valve from the delivery sheath 216 once the prosthetic valve has been advanced to a location at or adjacent the desired implantation location with the patient's body. For example, rotation of the first knob 211 in a first direction (for example, clockwise) can retract the sheath 216 proximally relative to the prosthetic valve 202 and rotation of the first knob 211 in a second direction (for example, counter-clockwise) can advance the sheath 216 distally. In some examples, the first knob 211 can be actuated by sliding or moving the first knob 211 axially, such as pulling and/or pushing the knob. In some examples, actuation of the first knob 211 (rotation or sliding movement of the first knob 211) can produce axial movement of the actuator assemblies 208 (and therefore the prosthetic valve 202) relative to the delivery sheath 216 to advance the prosthetic valve distally from the sheath 216.

The second knob 212 can be a rotatable knob configured to produce radial expansion and/or compression of the prosthetic valve 202. For example, rotation of the second knob 212 can rotate the threaded rods of the prosthetic valve 202 via the actuator assemblies 208. Rotation of the second knob 212 in a first direction (for example, clockwise) can radially expand the prosthetic valve 202 and rotation of the second knob 212 in a second direction (for example, counter-clockwise) can radially collapse the prosthetic valve 202. In some examples, the second knob 212 can be actuated by sliding or moving the second knob 212 axially, such as pulling and/or pushing the knob.

The third knob 214 can be a rotatable knob operatively connected to a proximal end portion of each actuator assembly 208. The third knob 214 can be configured to retract an outer sleeve or support tube of each actuator assembly 208 to disconnect the actuator assemblies 208 from the proximal portions of the actuators of the prosthetic valve (for example, threaded rod). Once the actuator assemblies 208 are uncoupled from the prosthetic valve 202, the delivery apparatus 200 can be removed from the patient, leaving just the prosthetic valve 202 in the patient.

Referring to FIGS. 4-5, they illustrate how each of the threaded rods 126 of the prosthetic device 100 can be removably coupled to an exemplary actuator assembly 300 (for example, actuator assemblies 208) of a delivery apparatus (for example, delivery apparatus 200). FIG. 5 illustrates how one of the threaded rods 126 can be coupled to an actuator assembly 300, while FIG. 4 illustrates how the threaded rod 126 can be detached from the actuator assembly 300.

As introduced above, an actuator assembly 300 can be coupled to the head portion 131 of each threaded rod 126. The head portion 131 can be included at a proximal end portion 180 of the threaded rod 126 and can extend proximally past a proximal end of the second post 124 (FIG. 2A). The head portion 131 can comprise first and second protrusions 182 defining a channel or slot 184 between them, and one or more shoulders 186. As discussed above, the head portion 131 can have a width greater than a diameter of the inner bore of the second post 124 such that the head portion 131 is prevented from moving into the inner bore of the second post 124 and such that the head portion 131 abuts the outflow end 108 of the frame 102. In some examples, the head portion 131 can abut an outflow apex 119b of the frame 102. The head portion 131 can be used to apply a distally-directed force to the second post 124, for example, during radial expansion of the frame 102.

Each actuator assembly 300 can comprise a first actuation member configured as a support tube or outer sleeve 302 and a second actuation member configured as a driver 304. The driver 304 can extend through the outer sleeve 302. The outer sleeve 302 is shown transparently in FIGS. 4-5 for purposes of illustration. The distal end portions of the outer sleeve 302 and driver 304 can be configured to engage or abut the proximal end of the threaded rod 126 (for example, the head portion 131) and/or the frame 102 (for example, the apex 119b). The proximal portions of the outer sleeve 302 and driver 304 can be operatively coupled to the handle of a delivery apparatus (for example, handle 204). The delivery apparatus in this example can include the same features described previously for delivery apparatus 200. In some examples, the proximal end portions of each driver 304 can be operatively connected to the knob 212 such that rotation of the knob 212 (clockwise or counterclockwise) causes corresponding rotation of the drivers 304. The proximal end portions of each outer sleeve 302 can be operatively connected to the knob 214 such that rotation of the knob 214 (clockwise or counterclockwise) causes corresponding axial movement of the sleeves 302 (proximally or distally) relative to the drivers 304. In some examples, the handle can include electric motors for actuating these components.

The distal end portion of the driver 304 can comprise a central protrusion 306 configured to extend into the slot 184 of the threaded rod 126, and one or more flexible elongated elements or arms 308 including protrusions or teeth 310 configured to be releasably coupled to the shoulders 186 of the threaded rod 126. The protrusions 310 can extend radially inwardly toward a longitudinal axis of the second actuation member 304. As shown in FIGS. 4-5, the elongated elements 308 can be configured to be biased radially outward to an expanded state, for example, by shape setting the elements 308.

As shown in FIG. 5, to couple the actuator assembly 300 to the threaded rod 126, the driver 304 can be positioned such that the central protrusion 306 is disposed within the slot 184 (FIG. 4) and such that the protrusions 310 of the elongated elements 308 are positioned distally to the shoulders 186. As the outer sleeve 302 is advanced (for example, distally) over the driver 304, the sleeve 302 compresses the elongated elements 308 they abut and/or snap over the shoulders 186, thereby coupling the actuator assembly 300 to the threaded rod 126. Thus, the outer sleeve 302 effectively squeezes and locks the elongated elements 308 and the protrusions 310 of the driver 304 into engagement with (i.e., over) the shoulders 186 of the threaded rod 126, thereby coupling the driver 304 to the threaded rod 126.

Because the central protrusion 306 of the driver 304 extends into the slot 184 of the threaded rod 126 when the driver 304 and the threaded rod 126 are coupled, the driver 304 and the threaded rod 126 can be rotational locked such that they co-rotate. So coupled, the driver 304 can be rotated (for example, using knob 212 the handle of the delivery apparatus 200) to cause corresponding rotation of the threaded rod 126 to radially expand or radially compress the prosthetic device. The central protrusion 306 can be configured (for example, sized and shaped) such that it is advantageously spaced apart from the inner walls of the outer sleeve 302, such that the central protrusion 306 does not frictionally contact the outer sleeve 302 during rotation. Though in the illustrated example the central protrusion 306 has a substantially rectangular shape in cross-section, in some examples, the protrusion 306 can have any of various shapes, for example, square, triangular, oval, etc. The slot 184 can be correspondingly shaped to receive the protrusion 306.

The outer sleeve 302 can be advanced distally relative to the driver 304 past the elongated elements 308, until the outer sleeve 302 engages the frame 102 (for example, a second post 124 of the frame 102). The distal end portion of the outer sleeve 302 also can comprise first and second support extensions 312 defining gaps or notches 314 between the extensions 312. The support extensions 312 can be oriented such that, when the actuator assembly 300 is coupled to a respective threaded rod 126, the support extensions 312 extend partially over an adjacent end portion (for example, the upper end portion) of one of the second posts 124 on opposite sides of the post 124. The engagement of the support extensions 312 with the frame 102 in this manner can counter-act rotational forces applied to the frame 102 by the rods 126 during expansion of the frame 102. In the absence of a counter-force acting against these rotational forces, the frame can tend to “jerk” or rock in the direction of rotation of the rods when they are actuated to expand the frame. The illustrated configuration is advantageous in that outer sleeves, when engaging the proximal posts 124 of the frame 102, can prevent or mitigate such jerking or rocking motion of the frame 102 when the frame 102 is radially expanded.

To decouple the actuator assembly 300 from the prosthetic device 100, the sleeve 302 can be withdrawn proximally relative to the driver 304 until the sleeve 302 no longer covers the elongated elements 308 of the driver 304. As described above, the sleeve 302 can be used to hold the elongated elements 308 against the shoulders 186 of the threaded rod 126 since the elongated elements 308 can be naturally biased to a radial outward position where the elongated elements 308 do not engage the shoulders 186 of the threaded rod 126. Thus, when the sleeve 302 is withdrawn such that it no longer covers/constrains the elongated elements 308, the elongated elements 308 can naturally and/or passively deflect away from, and thereby release from, the shoulders 186 of the threaded rod 126, thereby decoupling the driver 304 from the threaded rod 126.

The sleeve 302 can be advanced (moved distally) and/or retracted (moved proximally) relative to the driver 304 via a control mechanism (for example, knob 214) on the handle 204 of the delivery apparatus 200, by an electric motor, and/or by another suitable actuation mechanism. For example, the physician can turn the knob 214 in a first direction to apply a distally directed force to the sleeve 302 and can turn the knob 214 in an opposite second direction to apply a proximally directed force to the sleeve 302. Thus, when the sleeve 302 does not abut the prosthetic device and the physician rotates the knob 214 in the first direction, the sleeve 302 can move distally relative to the driver 304, thereby advancing the sleeve 302 over the driver 304. When the sleeve 302 does abut the prosthetic device, the physician can rotate the knob 214 in the first direction to push the entire prosthetic device distally via the sleeve 302. Further, when the physician rotates the knob 214 in the second direction the sleeve 302 can move proximally relative to the driver 304, thereby withdrawing/retracting the sleeve 302 from the driver 304.

Turning now to FIGS. 6A-11B, exemplary valvular structures including offset elements configured to be moveable, shiftable, slidable, and/or deformable from a neutral state or position to a radially outward shifted state or position are shown and described. FIGS. 6A and 6B show an exemplary valvular structure 400 (also referred to as a leaflet assembly), in a partially assembled state. The valvular structure 400 can be configured to be mounted within an annular frame of a prosthetic valve (such as, for example, the prosthetic valve 100 discussed above or other prosthetic valves). In some examples, the valvular structure 400 includes a plurality of leaflets 402 that can be coupled to the frame and can be configured to open and close to regulate flow of blood through the prosthetic valve. The leaflets 402 have leaflet free edges 414 (also referred to as coaptation edges) that move radially inward to coapt with each other and close the valve during systole and move radially outward to open the valve during diastole. The exemplary valvular structure 400 is shown as having three leaflets 402 that are arranged to collapse or coapt in a tricuspid arrangement. However, in some examples, the valvular structure 400 may have greater or fewer number of leaflets than three (for example, two leaflets, or four or more leaflets).

The leaflets 402 can be made of a flexible material. In some examples, the leaflets 402 may be made in whole or in part from pericardial tissue (for example, bovine pericardial tissue), biocompatible synthetic materials, or various other suitable natural or synthetic materials as known in the art and described in, for example, U.S. Pat. No. 6,730,118, which is incorporated herein by reference.

Each leaflet 402 includes primary tabs 406a, 406b on opposing sides of the leaflet and proximate to (or closer to) at an outflow end of the leaflet where the leaflet free edge 414 is located. In the partially assembled state shown in FIGS. 6A and 6B, the leaflets 402 may be partially secured together by sutures 404 at portions of their adjacent side edges (for example, at the side edge portions that extend below the primary tabs 406a, 406b, toward the inflow end of the leaflets). Suturing of adjacent sides as shown at 404 can be carried out before or after positioning the leaflets within a frame.

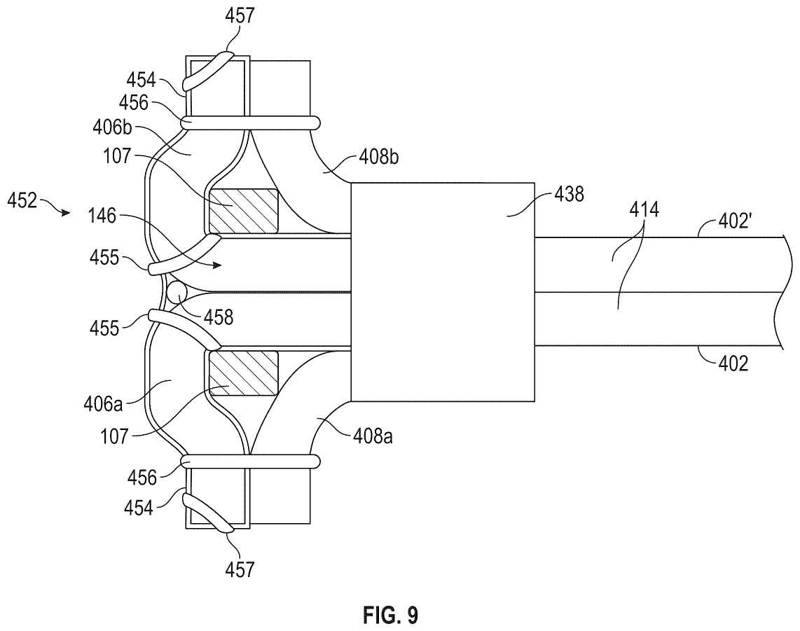

As shown in FIG. 6B, offset elements 460 are positioned over and aligned with side edges 418a, 418b proximate to an outflow end of adjacent leaflets 402, such that secondary tabs 408a, 408b of the offset elements 460 can be folded down and positioned adjacent to and in alignment with the primary tabs 406a, 406b of the leaflet 402. In this position, the secondary tabs 408a, 408b and the respective primary tabs 406a, 406b can cooperatively form commissures at the adjacent sides of the leaflets 402. The commissures can be mounted within or on a frame in a manner to secure the leaflets to the frame at least in part via the commissures (as illustrated in FIG. 9-11B and described in detail below, and as described above with respect to FIGS. 1A and 1B).

As illustrated in FIG. 6B, the offset elements 460 are configured to offset the leaflet free edges 414 from the primary tabs 406a, 406b and the side edges of the leaflets 402. The offset elements 460 can be made of a flexible material. In some examples, the offset elements 460 may be made in whole or in part from pericardial tissue (for example, bovine pericardial tissue), biocompatible synthetic materials, or various other suitable natural or synthetic materials as known in the art and described in, for example, U.S. Pat. No. 6,730,118, previously incorporated herein. In some examples, the offset elements 460 can be formed from any of various biocompatible fabrics (for example, PET fabric) or a non-fabric (non-textile) polymeric material, such as polyurethane.

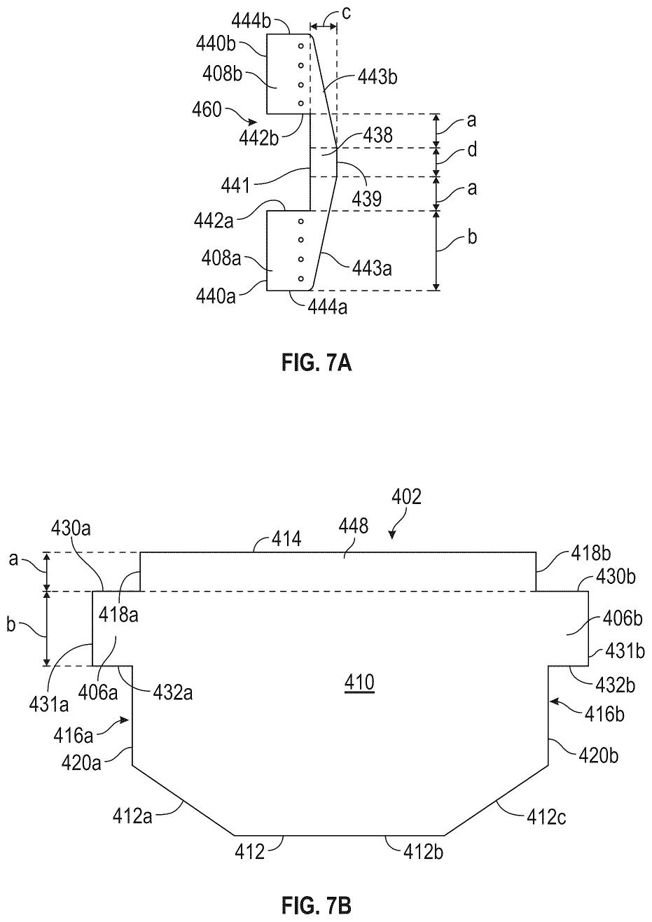

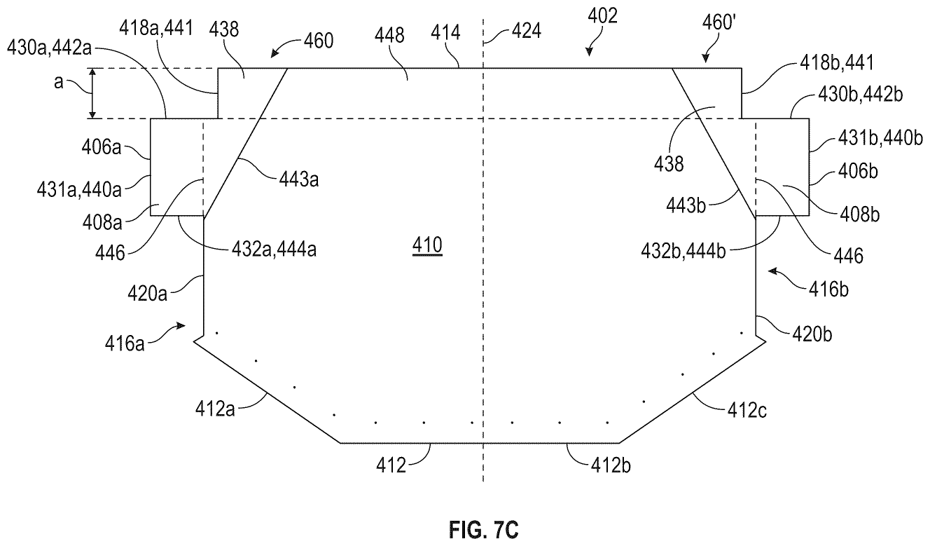

FIGS. 7A-7C illustrate the structure of the individual leaflets 402 and offset elements 460. As can be seen therein, the leaflet 402 can comprise a flexible sheet 410 forming a leaflet body. The flexible sheet 410 can comprise one or more of the leaflet materials previously discussed herein or other materials known in the art. In some examples, the thickness of the flexible sheet 410 can be in a range from 0.1 mm to 1 mm. The flexible sheet 410 can include a leaflet attachment edge 412, which can also be referred to as the inlet edge or cusp edge of the leaflet (the lower edge in the figure) and which opposes the leaflet free edge 414 (the upper edge in the figure). When the leaflet 402 is mounted within an annular frame as part of a valvular structure, the leaflet attachment edge 412 can be positioned in or proximate to an inflow end portion of the frame (such as, the inflow end 109 of the frame 102 illustrated in FIGS. 1A-2B), and the leaflet free edge 414 can be positioned in or proximate to an outflow end portion of the frame (such as, the outflow end 108 of the frame 102 illustrated in FIGS. 1A-2B). The terms “inflow” and “outflow” are related to the normal direction of blood flow through a prosthetic valve when implanted within a heart.

As shown in the illustrated example, the leaflet attachment edge 412 may have a truncated V-shape or tapered shape. For example, the leaflet attachment edge 412 can include linear edges 412a, 412b, 412c that are arranged to form the truncated V-shape or tapered shape. In some examples, the leaflet attachment edge can have a truncated V-shape or tapered shape having one or more curved edges or a combination of linear edges and curved edges. In some examples, the leaflet attachment edge can have other shapes, such as being linear, U-shaped, or scalloped-shaped.

In some examples, the leaflet free edge 414 may be a linear edge (as shown) or an approximately linear edge. The approximately linear edge may be formed by two or more linear edges with different inclinations (thereby forming a central angled portion wherein the edges meet) or by a curved edge with a slight curvature, such as a slight convex curvature outwardly curving from the leaflet body (the flexible sheet 410).

Also shown in FIGS. 7A-7C, the flexible sheet 410 can include opposing side edges 416a, 416b that extend axially between respective ends of the leaflet attachment edge 412 and the leaflet free edge 414. The axial direction of the leaflet 402 is indicated by longitudinal axis 424 (FIG. 7C), which is transverse to the leaflet attachment edge 412 and the leaflet free edge 414. In some examples, the longitudinal axis 424 can be an axis about which leaflet 402 is symmetrical. In some examples, the leaflets may be asymmetrical.

In some examples, the side edge 416a includes side edge portions 418a and 420a, which are spaced apart in the axial direction by the primary tab 406a and are generally parallel to the longitudinal axis 424, and the side edge 416b includes side edge portions 418b and 420b, which are spaced apart in the axial direction by the primary tab 406b and are generally parallel to the longitudinal axis 424. The side edges 420a, 420b (also referred to as sub-commissure edges of the leaflet) are connected to opposite ends of the leaflet attachment edge 412. The side edges 418a, 418b can be referred to as supra-commissure edges of the leaflet.

During assembly of the valvular structure, the side edge 420a may be sutured to a side edge 420b of an adjacent leaflet and the side edge 420b can be attached or sutured to a side edge 420a of another adjacent leaflet (such as via the sutures 404 shown in FIGS. 6A and 6B). In some examples, the attached side edges 420a, 420b can be sutured or attached to a frame of the prosthetic valve. In some examples, the side edges 418a, 418b are neither sutured to side edges of adjacent leaflets nor attached to a frame. Rather, the side edges 418a, 418b can form an edge or cutaway portion of an upper material portion 448 of the leaflet 402 that offsets the leaflet free edge 414 from the commissure formed (in part) by the primary tabs 406a, 406b.

In some examples, the leaflet 402 can have a curved cusp edge, or leaflet attachment edge, (for example, a U-shaped cusp edge) that extends between the primary tabs 406a, 406b, such as disclosed in U.S. Pat. No. 9,393,110, which is incorporated herein by reference. For example, a curved cusp edge can extend from a lower edge 432a of primary tab 406a to a lower edge 432b of primary tab 406b. In some examples, the leaflet can have straight or substantially straight sub-commissure edges (similar to edges 420a, 420b) and a curved cusp edge that extends from one of the sub-commissure edges to the other sub-commissure edge.

The leaflet 402 can be attached to a frame (for example, attached to struts of the frame) via suturing along the leaflet attachment edge 412, for example, in the manner described above and shown in FIG. 1A. To protect the leaflet material along the leaflet attachment edge 412 from tears, a reinforcement material may be provided in the area, such as a reinforcing member 125. The reinforcing member can be made of a tear resistant material that is biocompatible. In one example, the tear resistant material may be polyethylene terephthalate (PET), although various other synthetic or natural materials may be used. In general, the flexible sheet 410 at the leaflet attachment edge 412 may be strengthened by the reinforcement material without adding excessive bulk to the edge. As an example, the thickness of the reinforcing strip may be less than 6 mil (0.15 mm), less than 4 mil (0.1 mm), or less than 2 mil (0.05 mm). In some examples, the leaflet attachment edge 412 can be indirectly attached to the frame by an inner skirt. For example, the leaflet attachment edge 412 can be sutured to the inner skirt, which in turn can be sutured directly to struts of the frame, such as disclosed in U.S. Pat. No. 9,393,110, previously incorporated herein.

As noted above, the leaflet 402 can further include the primary tabs 406a, 406b projecting from opposing side edges 416a, 416b of the flexible sheet 410, and the primary tabs 406a, 406b can be configured to cooperatively form commissures of the valvular structure with the secondary tabs 408a, 408b of the offset elements 460. In some examples, the primary tab 406a extends between the side edges 418a, 420a and projects outwardly relative to the side edges 418a, 420a, and the primary tab 406b extends between the side edges 418b, 420b and projects outwardly relative to the side edges 418b, 420b. The primary tabs 406a, 406b have first edges 430a, 430b (the top edges in the figures) and second edges 432a, 432b (the bottom edges in the figures). Each of the first edges 430a, 430b is axially offset (that is, offset in the axial direction of the leaflet) from the leaflet free edge 414 by the upper material portion 448 of the flexible sheet 410. The upper material portion 448 has a height a that defines the distance between the leaflet free edge 414 and the primary tabs 406a, 406b. In some examples, the height a can be 0.1 mm to 3.0 mm. A height b, which corresponds to tab side edges 431a, 431b respectively extending between the first edges 430a, 430b and the second edges 432a, 432b, defines a height of the primary tabs 406a, 406b. In some examples, the height b can be 2.0 mm to 10.0 mm.

As shown in FIG. 7A, the offset elements 460 can include a central bridge portion 438 disposed between and connecting the secondary tabs 408a, 408b. In the illustrated example, each of the tabs 408b, 408b can has similar dimensions and/or overall structure and shape to the primary tabs 406a, 406b of the leaflet 402. The tabs 408a, 408b include first edges 442a, 442b and second edges 444a, 444b (opposing the first edges 442a, 442b), and tab side edges 440a, 440b respectively extending between the first edges 442a, 442b and the second edges 444a, 444b. Each of the secondary tabs 408a, 408b has a height b, which can be approximately equal to the height b of the primary tabs 406a, 406b of the leaflet 402.

The bridge portion 438 can have an overall trapezoidal shape defined by an interior edge 439, an opposing exterior edge 441, and inclined edges 443a, 443b that extend between the interior edge 439 and the second edges 444a, 444b of tabs 408a, 408b. The interior edge 439 is parallel to the exterior edge 441 is configured to be interior relative to the edge 441 when the offset element 460, 460′ is attached to an adjacent pair of leaflets 402. Opposing end sections of the bridge portion 438 that extend between the first edges 442a, 442b and ends of the interior edge 439 have a height a, which can be approximately equal to the height a of the upper material portion 448 of the leaflet 402. A central section of the bridge portion 438 has a width c and a height d (where the height d corresponds to a length of the interior edge 439). In some examples, the width c can be 0.5 mm to 4.0 mm and the height d can be 0.1 mm to 1.0 mm. In some examples, as illustrated in FIG. 7A, the secondary tabs 408a, 408b form opposing end portions of the offset element 460 and extend outwards from the exterior edge 441 at or proximate to opposite ends of the bridge portion 438.

When being assembled into the valvular structure (for example, as in FIG. 7C), each of the offset elements 460, 460′ can be aligned with and folded over a portion of the upper material portions 448, such as over the corners of the upper material portions, of a pair of adjacent leaflets 402. In some examples, the secondary tab 408a of the offset element 460 can be positioned on the same side of the leaflet 402 as the primary tab 406a (left side in the figure) and form a cooperating pair with the primary tab 406a. In some examples, the secondary tab 408b of the offset element 460′ can be positioned on the same side of the leaflet 402 as the primary tab 406b (right side in the figure) and form a cooperating pair with the primary tab 406b.

As can be seen in FIG. 7C, when the offset element 460 is in the aligned and folded position or configuration, the secondary tab 408a can be positioned over the primary tab 406a such that the edges 442a, 440a, 444a of the secondary tab 408a are respectively aligned with the edges 430a, 431a, 432a of the primary tab 406a. Further, when the offset element 460′ is in the aligned and folded position, the secondary tab 408b can be positioned over the primary tab 406b such that the edges 442b, 440b, 444b of the secondary tab 408b are respectively aligned with the edges 430b, 431b, 432b of the primary tab 406b. Yet further, each of the inclined edges 443a, 443b can form a continuous inclined edge that extends over surface of the leaflet 402 between the free edge 414 and the side edge 420a, 420b, respectively. For example, the inclined edges 443a, 443b can respectively extend between the leaflet free edge 414 and a point or location where the side edge portions 420a, 420b intersect with the second (bottom) edges 432a, 432b of the primary tabs 406a, 406b.

Also illustrated in FIG. 7C, when the offset elements 460, 460′ are in the aligned and folded position, the edges 430a, 442a and the edges 430b, 442b, are axially offset (i.e., offset in the axial direction of the leaflet) from leaflet free edge 414 by a distance a. In some examples, the distance a can be approximately equal to the height a of each of the upper material portion 448 of the leaflet 402 and the height a of the end sections of the bridge portion 438 of the offset elements 460, 460′. Further, in some examples, the outflow corner edge 418a can be aligned with the exterior edge 441 of the bridge portion 438 of the offset element 460, and the opposing outflow corner edge 418b can be aligned with the exterior edge 441 of the bridge portion 438 of the offset element 460′.

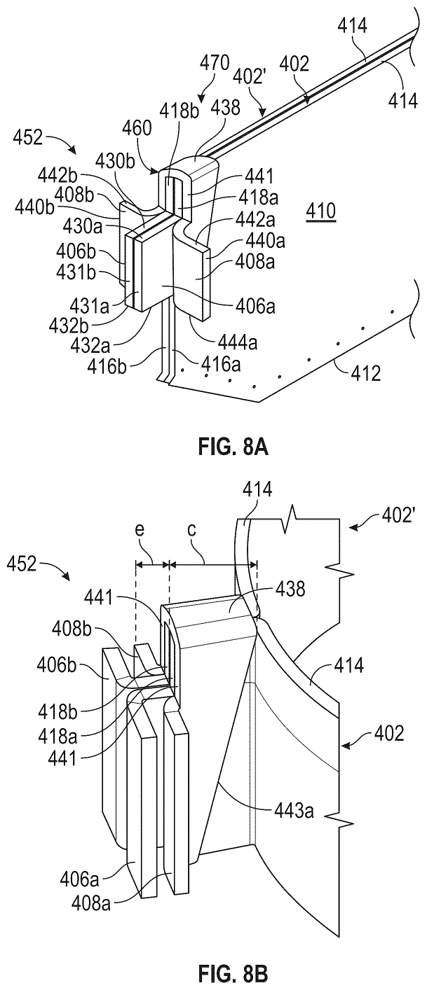

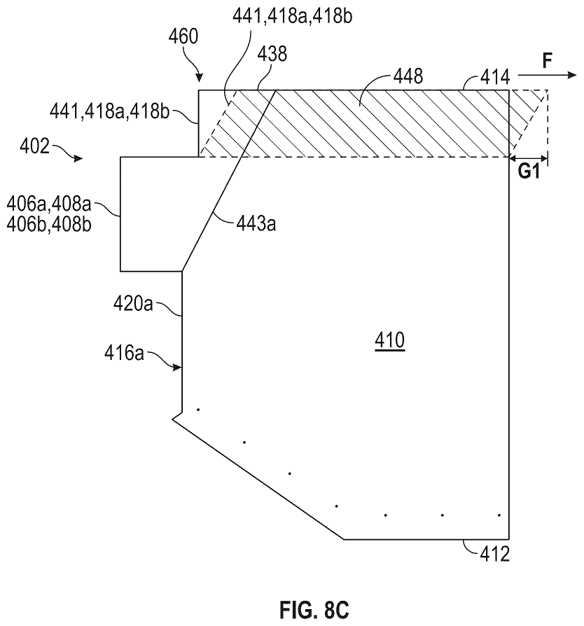

Forming of an exemplary commissure 452 between a pair of adjacent leaflets 402, 402′ and one of the offset elements 460 is illustrated in FIGS. 8A and 8B. As can be seen therein, the offset clement 460 is folded over an end portion of each of the upper material portions 448 (including the free edges 414) of the leaflets 402, 402′ such that the exterior edge 441 of the bridge portion 438 aligns with each of the edges 418a, 418b of the upper material portion 448. Further, the primary tabs 406a, 406b and the secondary tabs 408a, 408b can be folded about a vertical fold line 446 (shown in FIG. 7C) such that the paired primary and secondary tabs 406a, 408a and 406b, 408b are folded outward in a circumferential direction from the paired side edges 416a, 416b of the adjacent leaflets 402, 402′. In some examples, the fold lines 446 are coincident or aligned with the leaflet side edges 416a, 416b. As described herein, in an assembled configuration where the leaflets 402 of the valvular structure 400 are mounted within a frame (such as, for example, the frame 102 shown in FIGS. 1A-2B), the paired tabs 406a, 408a and 406b, 408b forming the commissure 452 can be attached to the frame.

When in the configuration shown in FIGS. 8A and 8B, the moveable portions of the free edges 414 of the leaflets 402, 402′ are laterally and/or radially offset (that is, offset in the lateral direction relative to the paired side edges 416a, 416b of the leaflet and/or offset radially from a frame of the prosthetic valve) by a distance c via the offset element 460. In some examples, when the valvular structure is in a neutral state or position (as shown in FIGS. 8A and 8B and discussed further below), the offset distance can be the same or substantially the same as the width c of the bridge portion 438 of the offset elements 460.

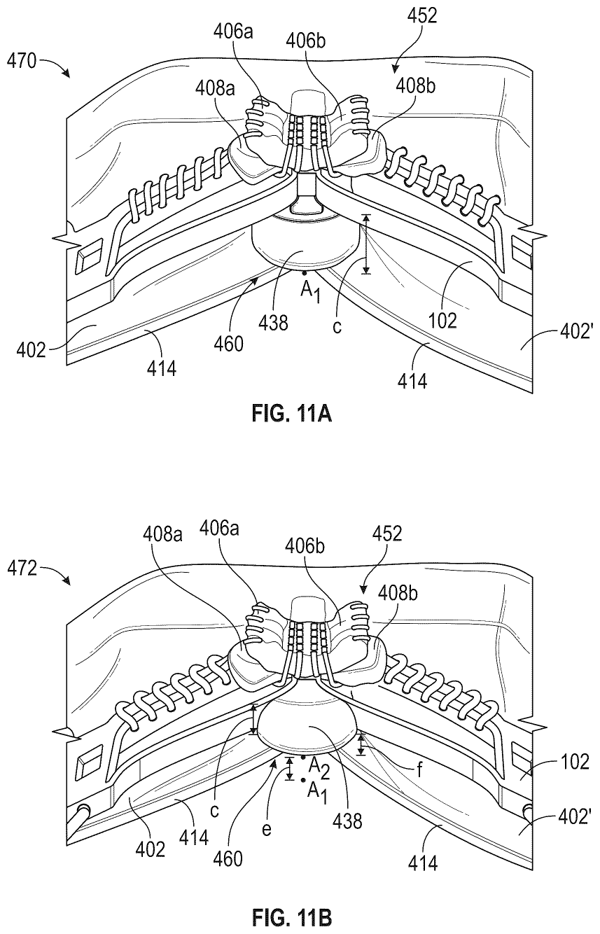

The upper material portion 448 and the bridge portion 438 of the offset element 460 are unattached to the frame, which means that the upper material portion 448 and the bridge portion 438 may be able to deflect or deform with the leaflet free edge 414 when the leaflet free edge 414 moves between open and closed states under the flow of blood (that is, working cycles of the prosthetic valve). The bridge portion 438 may be wrapped tightly around the upper material portion 448 such that frictional forces and/or compressive forces between the interior surface of the bridge portion 438 and the exterior surface of the upper material portions 448 retain a position of the bridge portion relative to the upper material portions of the adjacent leaflets (or at least limit or resist movement therebetween) under the pressure gradients across the prosthetic valve during the working cycles of the prosthetic valve. As described below in further detail below, in some examples, the bridge portion 438 can be configured to move, slide, or slip laterally and/or radially outward relative to the upper material portions by a distance e when subjected to a greater force acting thereon when the prosthetic valve is radially expanded the to a relatively larger working diameter. In other words, the bridge portion can be moved from the neutral position to a radially outward shifted position when the prosthetic valve is expanded to the relatively larger working diameter. In some examples, when the bridge portion is shifted to the radially outward shifted position, the bridge portion can be moved relative the frame of the prosthetic valve. For example, the bridge portion can be moved or positioned closer to the frame/commissure window of the prosthetic valve than when the bridge portion is the neutral position (see, for example, FIGS. 11A and 11B).

The upper material portion 448 and the bridge portion 438 can cooperatively provide a material slack to the leaflet free edge 414 that extends the radial reach of the leaflet free edge 414 during coaptation (during normal working cycles of the prosthetic valve). FIG. 8C illustrates cooperative deformation of the upper material portion 448 and the bridge portion 438 that can occur when the leaflets close under the backflow of blood (for a prosthetic aortic valve, this occurs during ventricular diastole). In the assembled state (when the leaflets are assembled as part of a valvular structure mounted within a frame), the upper material portions 448 of the leaflets 402 can extend generally vertically or axially relative to the commissures during systole. As the upper material portions 448 and the bridge portion 438 are unattached from the frame, they may be able to move radially inward (that is, towards the middle of the valve) in response to tension applied to the leaflet (indicated as F) during diastole and thus provides additional material for coaptation (identified as gain G1). The amount of material slack provided by the upper material portion 448 and the bridge portion 438 can depend at least in part on the height a (indicated in FIGS. 7A-7C), which is the amount by which the upper material portion 448 and the bridge portion 438 protrude relative to the commissures formed at the primary tabs 406a, 406b and the secondary tabs 408a, 408b when the leaflets are mounted in a frame.

In some examples, the height a can be selected to provide the material slack to achieve complete leaflet coaptation. In some examples, for valves using three leaflets and having diameters in a range from 20 mm to 40 mm (which, in some examples, is a minimum diameter and the valve can have an effective range of diameters, for example, up to an additional 4 mm), the height a can be in a range from 0.1 mm to 3.0 mm. In some examples, the height a in a range from 0.1 mm to 2.0 mm has been found to be effective for valve diameters in a range from 26 mm to 29 mm.

Since the upper material portion 448 can act to extend the reach of the leaflet for coaptation, in some examples, it can be possible to shorten the overall height of the leaflet and narrow the width of the leaflet to enable smaller valve sizes that meet desired coaptation and pressure gradient performance. Moreover, shortening the overall height of the leaflets can allow the free edges 414 of the leaflets to be spaced upstream of the outflow end of the frame of the prosthetic valve (see, for example, the frame 102 of FIGS. 1A-2B), which can minimize the risk of blocking the coronary ostia and can help preserve access to the coronary ostia during a subsequent valve-in-valve procedure.

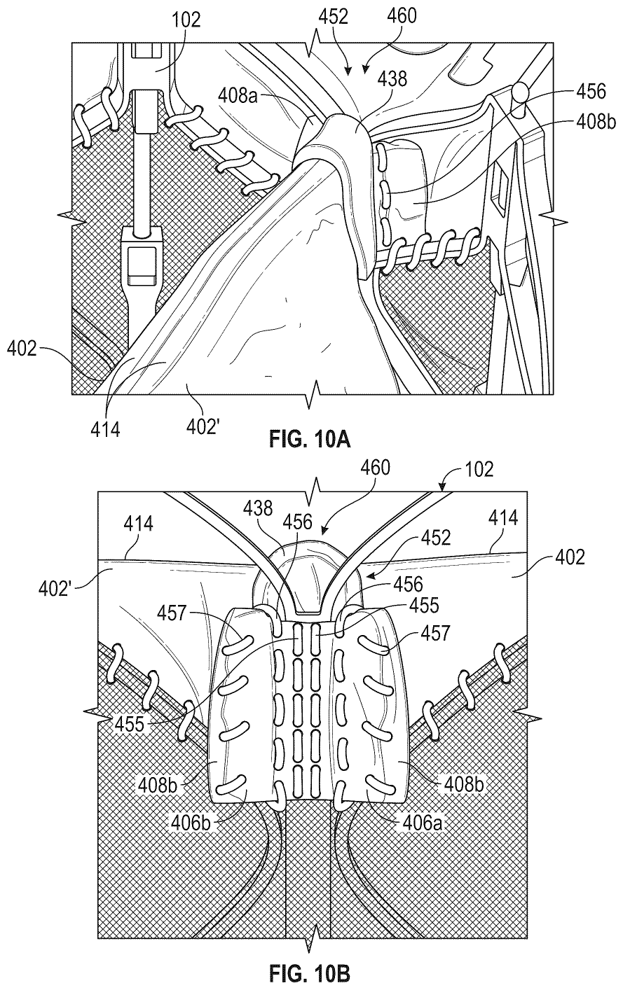

Turning to FIGS. 9-10B, exemplary suturing configurations for attachment of the paired tabs 406a, 408a and 406b, 408b at a commissure window 146 within a support post 107 of the frame 102 are illustrated. As can be seen therein, the primary tabs 406a, 406b can be inserted through the commissure window 146 and folded outwardly from the side edges of the leaflet such that they extend over exterior faces of adjacent struts 107 on opposing sides of the commissure window 146. The secondary tabs 408a, 408b can be folded outwardly from the opposing ends of the offset element 460 such that they extend over interior faces of adjacent struts 107 on opposing sides of the commissure window 146. Accordingly, an interior face of each of the primary tabs 406a, 406b can be aligned with, oriented toward, and/or abutted to a respective exterior face of each of the secondary tabs 408a, 408b.

In some examples, a flexible connector 454 can be connected to the pair of adjacent leaflets 402, 402′ at the primary tab 406a of the leaflet 402 and the primary tab 406b of the leaflet 402′ for forming the commissure 452. For example, as illustrated in FIG. 9, the flexible connector 454 wraps around the primary tabs 406a, 406b such that it covers the interior and exterior surfaces of each of the primary tabs and extends between the paired primary and secondary tabs 406a, 408a and between the paired primary and secondary tabs 406b, 408b. In some examples, the flexible connector 454 can be connected to the primary tabs 406a, 406b with sutures 455, 456, 457. The flexible connector 454 can function to protect the primary tabs or at least limit the primary tabs 406a, 406b from wear or weakening caused by contact with the strut 107 and/or to reinforce the stitches that secure the tabs 406a, 406b to the tabs 408a, 408b.

The flexible connector 454 can comprise, for example, a piece of fabric (for example, PET fabric). A wedge element 458 can be connected to an interior side of the flexible connector 454 and be disposed between the first and second primary tabs 406a, 406b on an exterior side of the commissure window 146. The wedge element 458 can comprise, for example, a relatively heavy gauge suture, such as a braided suture (for example, an Ethibond suture), or a piece of fabric. The wedge element 458 can function for positioning and/or retaining a position or contact between the flexible connector 454 and the first and second primary tabs 406a, 406b. In some examples, the flexible connector 454 and/or the wedge clement 458 can be excluded or optional.

Each of the primary tabs 406a, 406b can be secured to the corresponding secondary tab 408a, 408b via sutures 456. For example, each of the paired primary and secondary tabs 406a, 408a and 406b, 408b can be attached to each other via sutures 456 to secure the paired the tabs to the struts 107 of the commissure window 146. In some examples, each of the sutures 456 can extend through a first (exterior) layer of the connector 454, a primary tab 406a, 406b, a second (interior) layer of the connector 454, and a secondary tab 408a, 408b. The end portions of the suture material used to form the sutures lines 456 (or separate sutures) can be used to form whip stitches 457 at the outer edges of the primary tabs 406a, 406b.

The remaining commissure tab assemblies of the leaflet assembly can be coupled to respective commissure windows 146 of the frame 102 (shown in FIG. 1A-2B) in the same manner as described above. Further details of the method for forming the commissure tab assemblies and coupling them to the frame that can be utilized with the valvular structures described herein are disclosed in U.S. Pat. No. 9,393,110, previously incorporated herein.

It should be noted that FIG. 9 shows one exemplary technique for coupling the commissures of a leaflet assembly to a frame. Other techniques, methods, and mechanisms can be used for coupling the commissures of the leaflet assembly to a frame, such as any of those disclosed in U.S. Pat. No. 9,393,110 (previously incorporated herein), U.S. Patent Application Publication No. 2018/0325665, or U.S. Patent Application No. 63/003,085, filed Mar. 31, 2020, which are each incorporated by reference herein.

As discussed above, the adjacent sub-commissure edges 420a, 420b (shown in FIGS. 7A and 7B) of adjacent leaflets 402, 402′ can be connected to each other with sutures 404 (shown in FIG. 6A and 6B). The sutures 404 can be, for example, in-and-out stitches or whip stitches that extend through a pair of adjacent sub-commissure edges 420a, 420b.

As discussed above with respect to FIGS. 8A-8C, the upper material portion 448 and the bridge portion 438 can be unattached to each other and unattached to the frame 102. Therefore, the upper material portions 448 and the bridge portion 438 can each be moveable or deformable relative to each other and to the frame 102. For example, as discussed above, FIG. 8C illustrates the cooperative deformation of the upper material portion 448 and the bridge portion 438 that can occur during diastole (under working cycles of the prosthetic valve).

In some examples, the bridge portion 438 can be moveable or deformable relative to the frame 102 and the upper material portion 448. For example, FIG. 8B illustrates a location of the bridge portion 438 when the offset element is in a neutral state or position. In the neutral state or position, the exterior edge 441 of the bridge portion can be aligned with the side edges 418a, 418b of the upper material portion 448, and an offset distance of an articulation axis for the leaflets 402, 402′ can be equal to the width c of the bridge portion 438. As the bridge portion 438 is deformable or moveable, it can be selectively shifted outwardly by a distance e and moved into a radially outward shifted position or state. In the radially outward shifted position or state, an offset distance of the articulation axis for the leaflets 402, 402′ can be equal to the width c of the bridge portion 438 less the outward shifted distance e. In some examples, the distance e can be 0.2 mm to 2.0 mm.

Transition of the bridge portion 438 of the offset element 460 from the neutral position or state to the radially outward shifted position or state is further illustrated in FIGS. 11A and 11B. As can be seen in FIG. 11A, in a neutral position 470 of the bridge portion 438, an articulation axis Aj of the leaflets 402, 402′ can be offset from the frame 102 by the distance c. As discussed above, the distance c can be substantially or generally equal to the width of the bridge portion 438. Although only one of the commissures 452 is shown in FIG. 11A, it will be appreciated that other the bridge portion of other offset elements in the prosthetic valve can be in a similar neutral position.

In some examples, the prosthetic valve illustrated in FIG. 11A is radially expanded to a diameter smaller than the nominal working diameter of the prosthetic valve. For example, a 29-mm prosthetic valve can be expanded to 27 mm. Thus, the offset distance c can be a sufficient or optimal distance where the articulation axes Aj of the leaflets 402 are positioned radially inwardly relative to the frame 102 to prevent or reduce contact between the leaflets and the frame during opening and closing (working cycles) of valvular structure when the prosthetic valve is expanded to the relatively smaller diameter. Further, the offset distance c can enable the leaflets to open to have an effective outflow area (EOA) that maintains appropriate pressure gradients across the prosthetic valve when operative at the relatively smaller diameter.

The prosthetic valve can have an operating range between the relatively smaller diameter and a relatively larger diameter. For example, the prosthetic valve can have a 4 mm operating range between a minimum diameter, such as 20 mm, 23 mm, or 27 mm, and a maximum diameter, such as, respectively, 24 mm, 27 mm, or 31 mm. Thus, the relatively larger diameter for the prosthetic valve can be up to 4.0 mm greater than the relative smaller diameter.

As illustrated in FIG. 11B, the bridge portion 438 can be transitioned from the neutral position 470 to a radially outward shifted position 472 by moving, sliding, or deforming the bridge portion 438 toward the frame 102 or away from a central axis of the prosthetic valve by a distance e.

In some examples, when the frame 102 of the prosthetic valve is radially expanded to the relatively larger diameter, the larger diameter can result in higher forces acting on the commissures, such that the bridge portion 438 of the offset element 460 can be moved or can slip radially outward relative to the upper material portion 448 (closer to the commissure window of the frame), thereby transitioning the offset elements to the radially outward shifted position. Additionally or alternatively, in some examples, the bridge portions 438 of the offset elements 460 can be moved by a post-implantation procedure. For example, if high gradients are indicated after implantation of the prosthetic valve, a post-ballooning procedure can be performed (for example, by inserting a balloon into the prosthetic valve and expanding the balloon to apply an outward radial force on the offset elements that will move the bridge portions thereof into the radially outward shifted position).

In the illustrated example of the radially outward shifted position, at least a portion the bridge portion 438 can extend over or overlap with at least a portion of the frame 102 such that a shifted articulation axis A₂ of the leaflets 402, 402′ is offset from the frame 102 by a distance f. The distance f can be substantially or generally equal to the distance c less the distance e. Although only one of the commissures 452 is shown in FIG. 11B, it will be appreciated that, in some examples, bridge portions of the other offset elements in the prosthetic valve can be moved into a similar radially outward shifted position. In some examples, the bridge portions of others of the offset elements can remain in the neutral position or can be adjusted to a radially outward shifted position having a different configuration relative to the position illustrating in FIG. 11B (for example, a bridge portion can be radially shifted outward to a lesser degree).

In some examples, the prosthetic valve illustrated in FIG. 11B is radially expanded to a relatively larger diameter (in other words, a diameter that is larger than the smaller diameter), such as, for example, a diameter in a range of 28 mm to 31 mm. The offset distance f can be a sufficient or optimal distance where the articulation axes A₂ of the leaflets 402 are positioned radially inwardly relative to the frame 102 to prevent or reduce contact between the leaflets and the frame during opening and closing of the prosthetic valve when the valve is expanded to the relatively larger diameter. Further, the offset distance f can enable the leaflets to open to have an effective outflow area (EOA) that maintains appropriate pressure gradients across the prosthetic valve when expanded to the relatively larger diameter.

Thus, the neutral position of an offset element (such as shown in FIG. 11A) can correspond to a first expanded diameter of a prosthetic valve and a radially outward shifted position of the offset element (such as shown in FIG. 11B) can correspond to a second expanded diameter of the prosthetic valve, which is greater than the first expanded diameter.

As discussed above, a degree or a diameter of radial expansion of the prosthetic valve can be dependent on anatomy of a patient, a disease condition of the native valve, a location of implantation of the prosthetic valve, and/or other factors. Further, a prosthetic valve can have an operating range between a relatively smaller diameter and a relatively larger maximum diameter. Accordingly, the valvular structure including the offset elements disclosed herein can be configured such that the offset distance for the articulation axis of the leaflets is adjustable or selectable to accommodate various diameters of radial expansion of a prosthetic valve. Thus, the prosthetic valves disclosed herein can enable use of the valve across a broader range of patients and/or disease conditions than conventional prosthetic valves.

Implantation and Delivery Techniques

As discussed above, once a prosthetic valve (such as, a prosthetic valve including the valvular structure 400 disclosed herein) is connected to a delivery apparatus (such as, the delivery apparatus 200), the prosthetic valve can be placed in a radially compressed state for delivery into a patient's body. In some examples, the compressed prosthetic valve can be loaded into a delivery capsule of the delivery apparatus or can be maintained in a compressed state without a delivery capsule. Thereafter, the delivery apparatus containing the prosthetic valve can be inserted into the patient's vasculature and advanced to the desired implantation site (for example, one of the native heart valves). Various delivery techniques for delivering the prosthetic valve to various implantation sites are described below. In some examples, the delivery techniques and methods are carried out in a simulation procedure, which are not conducted on a living human body. For example, the methods and techniques can be performed on a model anatomical system, in a cadaver, or in an animal.

Once the prosthetic valve is positioned at the desired implantation site (and deployed from the delivery capsule if the delivery apparatus includes a delivery capsule), the prosthetic valve can be radially expanded via the step-wise or gradual radial expansion methods described above.

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by deploying the prosthetic valve from a sheath to allow the prosthetic valve to self-expand via the step-wise radial expansion methods discussed above). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) are introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Further, any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include gamma radiation, ultra-violet radiation, and/or electron beam. Examples of chemicals for use in sterilization include ethylene oxide, hydrogen peroxide, peracetic acid, formaldehyde, and/or glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve comprising: an annular frame; and a valvular structure comprising a plurality of leaflets positioned within the annular frame and a plurality of offset elements, the valvular structure having an inflow end and an outflow end opposing the inflow end; wherein each of the leaflets comprises: a leaflet free edge at the outflow end of the valvular structure; and first and second primary tabs projecting from opposite sides of the leaflet; wherein each of the offset elements comprises: a bridge portion folded over a portion of the leaflet free edges of a pair of adjacent leaflets of the plurality of leaflets; and a first secondary tab extending from a first end portion of the bridge portion and a second secondary tab extending from a second opposing end portion of the bridge portion; wherein the first secondary tab is paired with the first primary tab of a first one of the pair of adjacent leaflets and the second secondary tab paired with the second primary tab of a second one of the pair of adjacent leaflets to form a commissure of the valvular structure, the commissure attached to the annular frame; and wherein the bridge portion of each offset element is configured to be transitionable relative to the annular frame from a neutral position to a radially outward shifted position.

Example 2. The prosthetic valve of any example herein, particularly example 1, wherein, when the bridge portion is in the neutral position, an articulation axis for the pair of adjacent leaflets is radially offset a first distance from the annular frame.

Example 3. The prosthetic valve of any example herein, particularly example 2, wherein, when the bridge portion is in the radially outward shifted position, the articulation axis is radially offset a second distance from the annular frame, wherein the second distance is less than the first distance.

Example 4. The prosthetic valve of any example herein, particularly examples 2 or 3, wherein the first distance is equal to a width of the bridge portion.

Example 5. The prosthetic valve of any example herein, particularly examples 1-4, wherein the bridge portion of each of the offset elements is unattached from the annular frame.

Example 6. The prosthetic valve of any example herein, particularly examples 1-5, wherein each leaflet comprises an upper material portion defining the leaflet free edge, wherein opposing end portions of the upper material portion define first and second supra-commissure side edges of the leaflet.

Example 7. The prosthetic valve of any example herein, particularly example 6,wherein the upper material portion is unattached from the annular frame, the offset element, and other leaflets adjacent thereto.

Example 8. The prosthetic valve of any example herein, particularly examples 6 or 7, wherein the upper material portion and the bridge portion are deformable relative to the annular frame.

Example 9. The prosthetic valve of any example herein, particularly examples 6 -8, wherein the upper material portion and the bridge portion are configured to provide material slack for coaptation of the leaflet free edge with the free edges of other leaflets.

Example 10. The prosthetic valve of any example herein, particularly examples 6-9, wherein, when the bridge portion is in the neutral position, an exterior edge of the bridge portion is aligned with the first and second supra-commissure side edges of the adjacent pair of leaflets.

Example 11. The prosthetic valve of any example herein, particularly examples 6-10, wherein, when the bridge portion is in the radially outward shifted position, at least a portion of the exterior edge of the bridge portion is positioned closer toward the annular frame the first supra-commissure side edge of the first one of the pair of adjacent leaflets.

Example 12. The prosthetic valve of any example herein, particularly examples 6-11, wherein each of the first primary tab and the second primary tab is axially offset relative to the leaflet free edge by the upper material portion.

Example 13. The prosthetic valve of any example herein, particularly examples 1-12, wherein the first secondary tab paired with the first primary tab of the first one of the pair of adjacent leaflets and the second secondary tab paired with the second primary tab of the second one of the pair of adjacent leaflets are coupled to a commissure window of the annular frame.

Example 14. The prosthetic valve of any example herein, particularly example 13, wherein the first primary tab of the first one of the pair of adjacent leaflets is inserted through the commissure window and folded over an exterior surface of a first strut of the commissure window, and wherein the second primary tab of the second one of the pair of adjacent leaflets is inserted through the commissure window and folded over an exterior surface of a second strut of the commissure window.

Example 15. The prosthetic valve of any example herein, particularly example 14, wherein the first secondary tab is folded over an interior surface of the first strut of the commissure window, and wherein the second secondary tab is folded over an interior surface of the second strut of the commissure window.

Example 16. The prosthetic valve of any example herein, particularly example 15, wherein the first primary tab is sutured to the first secondary tab to secure the paired first primary tab and first secondary tab to the first strut of the commissure window, and wherein the second primary tab is sutured to the second secondary tab to secure the paired second primary tab and second secondary tab to the second strut of the commissure window.

Example 17. The prosthetic valve of any example herein, particularly examples 13-16, wherein an exterior face of the first secondary tab is oriented toward an interior face of the first primary tab, and wherein an exterior face of the second secondary tab is oriented toward an interior face of the second secondary tab.

Example 18. The prosthetic valve of any example herein, particularly examples 13-17, wherein the valvular structure further comprises a flexible connector that covers surfaces of the first primary tab and the second primary tab.

Example 19. The prosthetic valve of any example herein, particularly example 18, wherein the flexible connector is disposed between the first and second struts and the first and second primary tabs.

Example 20. The prosthetic valve of any example herein, particularly examples 13-20, wherein the valvular structure further comprises a wedge element disposed between the first primary tab and the second primary tab exterior of the commissure window.

Example 21. The prosthetic valve of any example herein, particularly examples 1-20, wherein each leaflet comprises first and second sub-commissural sides edges, wherein the first sub-commissure side edge of the first one of the pair of adjacent leaflets is sutured to the second sub-commissure side edge of the second one of the pair of adjacent leaflets.

Example 22. The prosthetic valve of any example herein, particularly examples 1-21, wherein the bridge portion is configured to remain in the neutral position when the prosthetic valve is radially expanded to a first expanded diameter, and wherein the bridge portion is further configured to move to the outward shifted position when the prosthetic valve is expanded to a second expanded diameter that is greater than the first expanded diameter.

Example 23. The prosthetic valve of any example herein, particularly example 22, wherein the second expanded diameter is up to 4 mm greater than the first expanded diameter.

Example 24. The prosthetic valve of any example herein, particularly examples 1-23, wherein the prosthetic valve has a minimum operational diameter in a range of 20 mm to 40 mm, wherein the prosthetic valve has an effective operating range from the minimum operational diameter up to an additional 4 mm.

Example 25. The prosthetic valve of any example herein, particularly examples 1-24, wherein the bridge portion is configured to be transitioned from the neutral position to the radially outward shifted position via exertion of an outward radial force on the offset elements by an inflated balloon in a post-implantation procedure.

Example 26. The prosthetic valve of any example herein, particularly examples 1-25, wherein one or more of frictional forces or compressive forces between an interior surface of the bridge portion and exterior surfaces of the portions of the leaflet free edges resist movement of the bridge portion relative to the pair of adjacent leaflets during working cycles of the prosthetic valve.

Example 27. The prosthetic valve of any example herein, particularly examples 1-26, wherein, when the bridge portion is in the neutral position, the prosthetic valve has a smaller effective outflow area relative to when the bridge portion is in the radially outward shifted position.

Example 28. The prosthetic valve of any example herein, particularly examples 1-27, wherein, when the bridge portion is in the radially outward shifted position, the prosthetic valve has a larger effective outflow area relative to when the bridge portion is in the neutral position.

Example 29. The prosthetic valve of any example herein, particularly examples 1-28, wherein the plurality of leaflets comprise pericardial tissue.

Example 30. The prosthetic valve of any example herein, particularly examples 1-29, wherein the plurality of offset elements comprise pericardial tissue.

Example 31. The prosthetic valve of any example herein, particularly examples 1-9, wherein the bridge portion has a trapezoidal shape comprising an interior edge, an exterior edge parallel to the interior edge, a first inclined edge, and a second inclined edge, wherein the interior edge is shorter than the exterior edge.

Example 32. The prosthetic valve of any example herein, particularly example 31, wherein an intersection between the first inclined edge and a lower edge of the first secondary tab defines a first end of the offset element and an intersection between the second inclined edge and a lower edge of the second secondary tab defines a second end of the offset element.

Example 33. The prosthetic valve of any example herein, particularly example 32, wherein the first secondary tab projects outwardly relative to the exterior edge of the bridge portion at or proximate to the first end of the offset element and the second secondary tab projects outwardly relative to the exterior edge of the bridge portion at or proximate to the second end of the offset element.

Example 34. The prosthetic valve of any example herein, particularly examples 31-33, wherein, when the bridge portion is in the neutral position, a distance between the interior edge and the exterior edge defines a first offset distance of the leaflet free edges of the first one of the pair of adjacent leaflets and the second one of the pair of adjacent leaflets relative to the annular frame.

Example 35. The prosthetic valve of any example herein, particularly example 34, wherein, when the bridge portion is in the radially outward shifted position, the distance between the interior edge and the exterior edge less a distance that the bridge portion is radially shifted outward defines a second offset distance of the leaflet free edges of the first one of the pair of adjacent leaflets and the second one of the pair of adjacent leaflets relative to the annular frame, wherein the first offset distance is greater than the second offset distance.

Example 36. The prosthetic valve of any example herein, particularly examples 1-35, wherein the plurality of leaflets comprises three leaflets.

Example 37. The prosthetic valve of any example herein, particularly examples 1-36, wherein the plurality of offset elements comprises three offset elements.

Example 38. The prosthetic valve of any example herein, particularly examples 1-37, wherein the annular frame is a mechanically expandable frame.

Example 39. The prosthetic valve of any example herein, particularly example 38, further comprising at least one actuator coupled to the frame, wherein the actuator causes the frame to radially expand from a radially compressed state upon actuation of the actuator.

Example 40. The prosthetic valve of any example herein, particularly examples 1-39, wherein the annular frame is an at least partially self-expandable frame.

Example 41. The prosthetic valve of any example herein, particularly examples 1-40, wherein the offset elements are separate pieces of material from the leaflets.

Example 42. A prosthetic valve comprising: an annular frame having an inflow end, an outflow end, a longitudinal axis defining an axial direction, and a plurality of commissure windows; and a valvular structure mounted within the annular frame, the valvular structure comprising a plurality of leaflets and a plurality of offset elements, the plurality of leaflets forming a plurality of commissures attached to the commissure windows; wherein each leaflet of the plurality of leaflets comprises: a leaflet attachment edge coupled to the annular frame; a leaflet free edge; and an upper material portion of a first height and defining the leaflet free edge, wherein the upper material portion offsets the leaflet free edge from the commissure in an axial direction by the selected height, wherein the upper material portion has opposing end regions, wherein each end region is paired with an adjacent end region of an adjacent leaflet; and wherein each offset element comprises: a bridge portion of a first width, wherein the bridge portion is wrapped over an adjacent pair of end regions of the upper material portions of adjacent leaflets, wherein an articulation axis of the adjacent leaflets is offset from the annular frame in a radial direction by the first width when the bridge portion is in a neutral position; and opposing end portions, each of the opposing end portions disposed at one end of the bridge portion and attached to one of the adjacent leaflets.

Example 43. The prosthetic valve of any example herein, particularly example 42, wherein the bridge portion is configured to be moveable from the neutral position to a radially outward shifted position.

Example 44. The prosthetic valve of any example herein, particularly example 43, wherein the articulation axis between the adjacent leaflets is offset from the annular frame in the radial direction by a second width, wherein the second width is equal to the first width less a distance that the bridge portion is moved when the bridge portion is transitioned to the radially outward shifted position.

Example 45. The prosthetic valve of any example herein, particularly examples 42-44, wherein each leaflet further comprises a first primary tab and a second primary tab projecting from respective opposing sides of the leaflet.

Example 46. The prosthetic valve of any example herein, particularly example 45, wherein the opposing end portions of the offset element comprise a first secondary tab and a second secondary tab projecting from respective opposing ends of an exterior edge of the bridge portion.

Example 47. The prosthetic valve of any example herein, particularly example 46, wherein the first primary tab forms a cooperating pair with the first secondary tab, and the second primary tab forms a cooperating pair with the second secondary tab.

Example 48. The prosthetic valve of any example herein, particularly example 47, wherein the first primary tab is sutured to the first secondary tab, and the second primary tab is sutured to the second secondary tab.

Example 49. The prosthetic valve of any example herein, particularly examples 46 or 47, wherein the paired first primary tab and first secondary tab and the paired second primary tab and second secondary tab form a commissure of the plurality of commissures.

Example 50. The prosthetic valve of any example herein, particularly examples 46-49, wherein the first and second secondary tabs are folded relative to the exterior edge of the bridge portion and extend circumferentially away from each other along an inner surface of a commissure window of the plurality of commissure windows.

Example 51. The prosthetic valve of any example herein, particularly example 50, wherein, the first and second primary tabs extend through the commissure window and are outwardly folded from a respective side edge of the one of the adjacent leaflets.

Example 52. The prosthetic valve of any example herein, particularly examples 42-51, wherein the selected height is in a range of 0.5 mm to 5.0 mm.

Example 53. The prosthetic valve of any example herein, particularly examples 42-52, wherein the first width is in a range of 0.5 mm to 4.0 mm.

Example 54. The prosthetic valve of any example herein, particularly examples 42-53, wherein the annular frame comprises a plurality of axially oriented frame posts, and wherein the plurality of commissure windows are formed in at least some of the plurality of axially oriented frame posts.

Example 55. The prosthetic valve of any example herein, particularly examples 42-54, wherein the bridge portion of the offset element is unattached from the annular frame.

Example 56. The prosthetic valve of any example herein, particularly examples 42-55, wherein the upper material portion and the bridge portion are configured to provide material slack for coaptation of the leaflet free edge with other free edges of the plurality of leaflets.

Example 57. The prosthetic valve of any example herein, particularly examples 42-56, wherein one or more of frictional forces or compressive forces between an interior surface of the bridge portion and exterior surface of the end region of the upper material portions of the adjacent leaflets resist movement of the bridge portion relative to the adjacent leaflets during working cycles of the prosthetic valve.

Example 58. A prosthetic valve comprising: an annular frame; and a valvular structure mounted within the annular frame, the valvular structure comprising a plurality of leaflets and a plurality of offset elements, first and second primary tabs projecting from sides of each of the plurality of leaflets and first and second secondary tabs projecting from ends of a bridge portion of each of the plurality of offset elements; wherein the first and second primary tabs of adjacent leaflets and the first and second secondary tabs of the plurality of offset elements form a plurality of commissures coupled to the annular frame; wherein each leaflet in the plurality of leaflets comprises an upper material portion comprising a free edge offset in an axial direction from the plurality of commissures and unattached to the annular frame; wherein an articulation axis between the adjacent leaflets at each of the plurality of commissures is offset in a radial direction from the annular frame by an offset distance; and wherein the bridge portion of each of the plurality of offset elements is unattached to the annular frame

Example 59. The prosthetic valve of any example herein, particularly example 58, wherein the upper material portion and the bridge portion contribute a material slack that extends a radial reach of the free edge for coaptation.

Example 60. The prosthetic valve of any example herein, particularly examples 58 or 59, wherein the offset distance at each of the commissures is defined by a position of the bridge portion of a respective one of the plurality of offset elements, and the bridge portion of each of the plurality of offset elements is configured to be moveable from a neutral position to a radially outward shifted position.

Example 61. The prosthetic valve of any example herein, particularly example 60, wherein, when the bridge portion is in the neutral position, the articulation axis is offset from the annular frame by a first distance.

Example 62. The prosthetic valve of any example herein, particularly example 61, wherein, when the bridge portion is in the radially outward shifted position, the articulation axis is offset from the annular frame by a second distance, the second distance less than the first distance.

Example 63. The prosthetic valve of any example herein, particularly examples 60-62, wherein the bridge portion is configured to remain in the neutral position when the prosthetic valve is radially expanded to a first expanded diameter, and wherein the bridge portion is further configured to move to the outward shifted position when the prosthetic valve is expanded to a second expanded diameter that is greater than the first expanded diameter.

Example 64. The prosthetic valve of any example herein, particularly example 63, wherein the second expanded diameter is up to 4 mm greater than the first expanded diameter.

Example 65. The prosthetic valve of any example herein, particularly examples 58-64, wherein the bridge portion extends over aligned supra-commissural portions of the upper material portions of the adjacent leaflets.

Example 66. The prosthetic valve of any example herein, particularly example 65, wherein one or more of frictional forces or compressive forces between an interior surface of the bridge portion and exterior surfaces of upper material portions of the adjacent leaflets resist movement of the bridge portion relative to the adjacent leaflets during working cycles of the prosthetic valve.

Example 67. A method of implanting a prosthetic valve comprising: inserting a distal end of a delivery apparatus into a vasculature of a patient, the distal end of the delivery apparatus having the prosthetic valve coupled thereto in a radially compressed state, the prosthetic valve comprising an annular frame and a valvular structure, the valvular structure comprising a plurality of leaflets and offset elements arranged to form a plurality of commissures, wherein the commissures form respective articulation axes for the leaflets that are offset from the annular frame by the offset elements, wherein at each commissure, one of the offset elements extends over portions of free edges of a pair of adjacent leaflets; advancing the prosthetic valve to a selected implantation site; and radially expanding the prosthetic valve from the radially compressed state to a radially expanded state, wherein the offset elements are in a neutral position after the prosthetic valve is radially expanded.

Example 68. The method of any example herein, particularly example 67, further comprising further radially expanding the prosthetic valve to a further radially expanded state, which cause the offset elements to move from the neutral position to a radially outward shifted position closer to the frame.

Example 69. The method of any example herein, particularly example 68, wherein the prosthetic valve in the further radially expanded has a diameter up to 4 mm greater than a diameter of the prosthetic in the radially expanded state.

Example 70. The method of any example herein, particularly example 69, wherein further radially expanding the prosthetic valve comprises inflating a balloon positioned within the prosthetic valve.

Example 71. The method of any example herein, particularly example 69, wherein further radially expanding the prosthetic valve comprises actuating an actuator of the prosthetic valve to mechanically expand the prosthetic valve.

Example 72. The method of any example herein, particularly examples 67-71, wherein the method is a simulation implantation procedure.

Example 73. A prosthetic valve comprising: a radially compressible and expandable annular frame; and a valvular structure disposed inside of the frame, wherein the valvular structure comprises a plurality of leaflets and a plurality of offset elements arranged to form a plurality of commissures, wherein each leaflet comprises a coaptation edge, wherein each commissure comprises one of the offset elements wrapped over a portion of the coaptation edges of a pair of adjacent leaflets such that an articulation axis of the adjacent leaflets is offset radially inwardly from an inner surface of the frame, and wherein the offset elements are configured to be moveable relative to the annular frame from a neutral position to a radially outward shifted position.

Example 74. The prosthetic valve of any example herein, particularly example 73, wherein the prosthetic valve is radially expandable to a first expanded diameter and a second expanded diameter greater than the first expanded diameter, wherein when the prosthetic valve is expanded to the first expanded diameter, the offset elements are in the neutral position and when the prosthetic valve is expanded to the second expanded diameter, the offset elements are in the outward shifted position.

Example 75. The prosthetic valve of any example herein, particularly examples 73 or 74, wherein the articulation axis of each commissure is closer to the inner surface of the frame when the offset elements are in the outward shifted position.

Example 76. The prosthetic valve of any example herein, particularly examples 73-75, wherein the offset elements each comprise a moveable bridge portion and opposing end portions, wherein the opposing end portions are attached to the adjacent leaflets.

Example 77. The prosthetic valve of any example herein, particularly example 76, wherein a position of the articulation axis is defined by a position of the moveable bridge portion.

Example 78. The prosthetic valve of any example herein, particularly examples 73-77, the coaptation edges of the leaflets and the offset elements are deformable relative to the annular frame and are configured to provide material slack for coaptation of the leaflets.

Example 79. The prosthetic valve of any example herein, particularly examples 73-78, wherein the prosthetic valve has a minimum operational diameter in a range of 20 mm to 40 mm, and wherein the prosthetic valve has an effective operating range from the minimum operational diameter up to an additional 4 mm.

Example 80. The prosthetic valve of any example herein, particularly examples 73-79, wherein, when the offset elements are in the neutral position, the prosthetic valve has a smaller effective outflow area relative to when the offset elements are in the radially outward shifted position.

Example 81. The prosthetic valve of any example herein, particularly examples 73-80, wherein, when the offset elements are in the radially outward shifted position, the prosthetic valve has a larger effective outflow area relative to when the offset elements are in the neutral position.

Example 82. The prosthetic valve of any example herein, particularly examples 73-81, wherein the plurality of offset elements comprise pericardial tissue.

Example 83. The prosthetic valve of any example herein, particularly examples 73-81, wherein the plurality of offset elements comprise biocompatible fabric.

Example 84. The prosthetic valve of any example herein, particularly examples 73-83, wherein the plurality of leaflets comprises three leaflets.

Example 85. The prosthetic valve of any example herein, particularly examples 73-84, wherein the plurality of offset elements comprises three offset elements.

Example 86. The prosthetic valve of any example herein, particularly examples 73-85, wherein the annular frame is a mechanically expandable frame.

Example 87. The prosthetic valve of any example herein, particularly examples 73-86, wherein the annular frame is an at least partially self-expandable frame.

Example 88. The prosthetic valve of any example herein, particularly examples 73-87, wherein the offset elements are separate pieces of material from the leaflets.

The features described herein with regard to any example can be combined with other features described in any one or more of the other examples, unless otherwise stated. For example, any one or more of the features of one locking mechanism can be combined with any one or more features of another locking mechanism. As another example, any one or more features of one prosthetic valve can be combined with any one or more features of another prosthetic valve.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A prosthetic valve comprising:
an annular frame; and
a valvular structure comprising a plurality of leaflets positioned within the annular frame and a plurality of offset elements, the valvular structure having an inflow end and an outflow end opposing the inflow end;
wherein each of the leaflets comprises:
a leaflet free edge at the outflow end of the valvular structure; and
first and second primary tabs projecting from opposite sides of the leaflet;
wherein each of the offset elements comprises:
a bridge portion folded over a portion of the leaflet free edges of a pair of adjacent leaflets of the plurality of leaflets; and
a first secondary tab extending from a first end portion of the bridge portion and a second secondary tab extending from a second opposing end portion of the bridge portion;
wherein the first secondary tab is paired with the first primary tab of a first one of the pair of adjacent leaflets and the second secondary tab paired with the second primary tab of a second one of the pair of adjacent leaflets to form a commissure of the valvular structure, the commissure attached to the annular frame; and
wherein the bridge portion of each offset element is configured to be transitionable relative to the annular frame from a neutral position to a radially outward shifted position.

2. The prosthetic valve of claim 1, wherein, when the bridge portion is in the neutral position, an articulation axis for the pair of adjacent leaflets is radially offset a first distance from the annular frame.

3. The prosthetic valve of claim 2, wherein, when the bridge portion is in the radially outward shifted position, the articulation axis is radially offset a second distance from the annular frame, wherein the second distance is less than the first distance.

4. The prosthetic valve of claim 2, wherein the first distance is equal to a width of the bridge portion.

5. The prosthetic valve of claim 1, wherein the bridge portion of each of the offset elements is unattached from the annular frame.

6. The prosthetic valve of claim 1, wherein each leaflet comprises an upper material portion defining the leaflet free edge, wherein opposing end portions of the upper material portion define first and second supra-commissure side edges of the leaflet.

7. The prosthetic valve of claim 6, wherein the upper material portion is unattached from the annular frame, the offset element, and other leaflets adjacent thereto.

8. The prosthetic valve of claim 6, wherein the upper material portion and the bridge portion are deformable relative to the annular frame.

9. The prosthetic valve of claim 6, wherein the upper material portion and the bridge portion are configured to provide material slack for coaptation of the leaflet free edge with the free edges of other leaflets.

10. The prosthetic valve of claim 6, wherein, when the bridge portion is in the radially outward shifted position, at least a portion of the exterior edge of the bridge portion is positioned closer toward the annular frame than the first supra-commissure side edge of the first one of the pair of adjacent leaflets.

11. The prosthetic valve of claim 1, wherein the first secondary tab paired with the first primary tab of the first one of the pair of adjacent leaflets and the second secondary tab paired with the second primary tab of the second one of the pair of adjacent leaflets are coupled to a commissure window of the annular frame.

12. The prosthetic valve of claim 1, wherein the bridge portion is configured to remain in the neutral position when the prosthetic valve is radially expanded to a first expanded diameter, and wherein the bridge portion is further configured to move to the outward shifted position when the prosthetic valve is expanded to a second expanded diameter that is greater than the first expanded diameter.

13. The prosthetic valve of claim 1, wherein the prosthetic valve has a minimum operational diameter in a range of 20 mm to 40 mm, wherein the prosthetic valve has an effective operating range from the minimum operational diameter up to an additional 4 mm.

14. The prosthetic valve of claim 1, wherein the bridge portion has a trapezoidal shape comprising an interior edge, an exterior edge parallel to the interior edge, a first inclined edge, and a second inclined edge, wherein the interior edge is shorter than the exterior edge.

15. A method of implanting a prosthetic valve comprising:
inserting a distal end of a delivery apparatus into a vasculature of a patient, the distal end of the delivery apparatus having the prosthetic valve coupled thereto in a radially compressed state, the prosthetic valve comprising an annular frame and a valvular structure, the valvular structure comprising a plurality of leaflets and offset elements arranged to form a plurality of commissures, wherein the commissures form respective articulation axes for the leaflets that are offset from the annular frame by the offset elements, wherein at each commissure, one of the offset elements extends over portions of free edges of a pair of adjacent leaflets;
advancing the prosthetic valve to a selected implantation site; and
radially expanding the prosthetic valve from the radially compressed state to a radially expanded state, wherein the offset elements are in a neutral position after the prosthetic valve is radially expanded.

16. A prosthetic valve comprising:
a radially compressible and expandable annular frame; and
a valvular structure disposed inside of the frame, wherein the valvular structure comprises a plurality of leaflets and a plurality of offset elements arranged to form a plurality of commissures, wherein each leaflet comprises a coaptation edge,
wherein each commissure comprises one of the offset elements wrapped over a portion of the coaptation edges of a pair of adjacent leaflets such that an articulation axis of the adjacent leaflets is offset radially inwardly from an inner surface of the frame, and wherein the offset elements are configured to be moveable relative to the annular frame from a neutral position to a radially outward shifted position.

17. The prosthetic valve of claim 16, wherein the prosthetic valve is radially expandable to a first expanded diameter and a second expanded diameter greater than the first expanded diameter, wherein, when the prosthetic valve is expanded to the first expanded diameter, the offset elements are in the neutral position and, when the prosthetic valve is expanded to the second expanded diameter, the offset elements are in the outward shifted position.

18. The prosthetic valve of claim 16, wherein the articulation axis of each commissure is closer to the inner surface of the frame when the offset elements are in the outward shifted position.

19. The prosthetic valve of claim 16, wherein the offset elements each comprise a moveable bridge portion and opposing end portions, wherein the opposing end portions are attached to the adjacent leaflets.

20. The prosthetic valve of claim 16, wherein the prosthetic valve has a minimum operational diameter in a range of 20 mm to 40 mm, and wherein the prosthetic valve has an effective operating range from the minimum operational diameter up to an additional 4 mm.
